SEQUENCE LISTING (30 sequences)

SEQ ID NO: 1 - DNA, 489 bp, Homo sapiens
  CDS (1)...(486)
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaacgggaatgtaacagaatctggatgcaaagaatgtgaggaactggaggaaaaaaatattaaagaatttttgcagagttttgtacatattgtccaaatgttcatcaacacttcttga

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - DNA, 489 bp, Artificial Sequence
  Synthetically generated oligonucleotide
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaacgggaatgtaacagaatctggatgcaaagaatgtgaggaactggaggaaaaaaatattaaagaatttttggacagttttgtacatattgtcgacatgttcatcaacacttcttga

SEQ ID NO: 4 - protein, 162 aa, Artificial Sequence
  Synthetically generated peptide
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTS

SEQ ID NO: 5 - protein, 24 aa, Homo sapiens
MPMGSLQPLATLYLLGMLVASCLG

SEQ ID NO: 6 - protein, 346 aa, Artificial Sequence
  Synthetically generated peptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 9 aa, Artificial Sequence
  Synthetically generated peptide
LLELQVISL

SEQ ID NO: 8 - protein, 5 aa, Artificial Sequence
  Synthetically generated peptide
ENLII

SEQ ID NO: 9 - DNA, 25 bp, Artificial Sequence
  Synthetically generatrd oligonucleotide
ggaattcaactgggtgaatgtaata

SEQ ID NO: 10 - DNA, 60 bp, Artificial Sequence
  Synthetically generated oligonucleotide
cgggatcctcaagaagtgttgatgaacatgtcgacaatatgtacaaaactgtccaaaaat

SEQ ID NO: 11 - DNA, 21 bp, Artificial Sequence
  Primer
gcctggatgctaacagaagga

SEQ ID NO: 12 - DNA, 20 bp, Artificial Sequence
  Primer
gttcatcccgtcgctatggt

SEQ ID NO: 13 - DNA, 24 bp, Artificial Sequence
  Probe sequence
atgacaaggatgccgggcaagtgt

SEQ ID NO: 14 - DNA, 21 bp, Artificial Sequence
  Primer
aatctgtggctaccggtggta

SEQ ID NO: 15 - DNA, 24 bp, Artificial Sequence
  Primer
ggtggaagagctgatacattccta

SEQ ID NO: 16 - DNA, 25 bp, Artificial Sequence
  Probe sequence
atggttctggtggctctggttggaa

SEQ ID NO: 17 - DNA, 23 bp, Artificial Sequence
  Primer
gcaaagactggcttcatatccat

SEQ ID NO: 18 - DNA, 21 bp, Artificial Sequence
  Primer
gcagaagaggtgttccattgg

SEQ ID NO: 19 - DNA, 26 bp, Artificial Sequence
  Probe sequence
acaaggaccagctctgtccttggcag

SEQ ID NO: 20 - DNA, 20 bp, Artificial Sequence
  Primer
tgctcttcgggaaccaagct

SEQ ID NO: 21 - DNA, 19 bp, Artificial Sequence
  Primer
cagggttgctgggcagtga

SEQ ID NO: 22 - DNA, 28 bp, Artificial Sequence
  Probe sequence
caccagagcagttctcaacctggacagc

SEQ ID NO: 23 - DNA, 23 bp, Artificial Sequence
  Primer
acaatgaacgctacacactgcat

SEQ ID NO: 24 - DNA, 21 bp, Artificial Sequence
  Primer
tggcagtaacagccagaaaca

SEQ ID NO: 25 - DNA, 25 bp, Artificial Sequence
  Probe sequence
ttggctttgcagctcttcctcatgg

SEQ ID NO: 26 - protein, 21 aa, Homo sapiens
MVLGTIDLCSCFSAGLPKTEA

SEQ ID NO: 27 - protein, 6 aa, Homo sapiens
EPKSCD

SEQ ID NO: 28 - protein, 6 aa, Artificial Sequence
  Synthetically generated peptide
DPKSAD

SEQ ID NO: 29 - protein, 48 aa, Homo sapiens
MRISKPHLRSISIQCYLCLLLNSHFLTEAGIHVFILGCFSAGLPKTEA

SEQ ID NO: 30 - protein, 232 aa, Artificial Sequence
  Synthetically generatrd peptide
DPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2006/0057102 A1
(43) Pub. Date: Mar. 16, 2006

(54) Mutant interleukin-15-containing compositions and suppression of an immune response

(76) Inventors: Xin Xiao Zheng, Wellesley, MA (US); Terry B. Strom, Brookline, MA (US); Thomas Moll, San Diego, CA (US)

Correspondence Address: FISH & RICHARDSON PC, P.O. BOX 1022, MINNEAPOLIS, MN, 55440-1022, US

(21) Appl. No.: 11/202,287

(22) Filed: Aug. 11, 2005

Related U.S. Application Data
(60) Provisional application No. 60/600,478, filed on Aug. 11, 2004.
(60) Provisional application No. 60/601,042, filed on Aug. 11, 2004.

(51) Int. Cl. A61K 38/20

(52) U.S. Cl. 424/85.2

(57) ABSTRACT

The invention features methods of treating patients who have received, or who are scheduled to receive, a heart, lung, or heart-lung transplant by administering to the patient an agent that antagonizes IL-15 or the IL-15 receptor (IL-15R).

### RELATED APPLICATIONS

This application claims the benefit of U.S. provisional applications 60/600,478 and 60/601,042, both filed Aug. 11, 2004. For the purpose of any U.S. patent that may issue from the present application, the entire contents of the prior provisional applications are hereby incorporated by reference.

### FUNDING

Some of the work described herein was supported by a grant from the National Institutes of Health. The United States government may therefore have certain rights in the invention.

### FIELD OF THE INVENTION

The field of the invention is cytokine-mediated therapeutics, particularly mutant IL-15-containing polypeptides that can be used, for example, to prolong~graft survival in, or otherwise improve the prognosis for, a transplant recipient.

### BACKGROUND

An effective immune response begins when an antigen or mitogen triggers the activation of T cells. T cell activation is accompanied by numerous cellular changes, including the expression of cytokines and cytokine receptors. One of the cytokines involved in the immune response is interleukin-15 (IL-15), a T cell growth factor that stimulates the proliferation and differentiation of B cells, T cells, natural killer (NK) cells, and lymphocyte-activated killer (LAK) cells in vitro (Lodolce et al., Immunity 9:669, 1998; Kennedy et al., J. Exp. Med. 191:771, 2000; for review see Fehninger and Caligiuri, Blood 97:14, 2001). In vivo, the proliferation of these cell types ensures an effective immune response. IL-15 exerts its influence by binding to a cell surface receptor that consists of three distinct subunits: an IL-2Rβ subunit, an IL-2Rγ subunit, and a unique IL-15Rα subunit. IL-15 binding is thought to stimulate activation of two receptor-associated kinases, Jak1 and Jak3 (Caliguiri, Blood 97:14, 2001). Jak1 and Jak3 activation results in phosphorylation of two signal transducer and activator of transcription (STAT) proteins, STAT3 and STAT5 (Caliguiri, Blood 97:14, 2001).

### SUMMARY

The present invention is based, in part, on our discovery that an antagonist of the IL-15 receptor (IL-15R) can prevent the rejection of fully vascularized murine heart allografts and induce antigen-specific tolerance. Accordingly, the invention features methods of treating patients who have received, or who are scheduled to receive, a heart, lung (or a portion thereof (e.g., a lobe or a portion of a lobe)), or heart-lung transplant by administering to the patient an agent that antagonizes IL-15 or the IL-15 receptor (IL-15R). The agent can be one that inhibits the expression or activity of IL-15 or one or more of the components of the IL-15R (i.e., one or more of the IL-2Rβ subunit, the IL-2Rγ subunit, and the IL-15Rα subunit). As the a subunit is unique to the receptor complex bound by IL-15, reducing the expression or activity of only the a subunit provides a treatment that does not affect (or is less likely to affect) IL-2R-bearing cells and/or IL-2R-mediated cellular activities. Similarly, administering an agent that targets the IL-15R to the substantial exclusion of the IL-2R is not expected to affect IL-2R-bearing cells and/or IL-2R-mediated cell processes.

While agents that can be used in the methods of the present invention are described further below, we note here that they include nucleic acids (whether DNA-based or RNA-based), polypeptides (including antibodies), and chemical compounds (e.g., small organic or inorganic compounds, such as those available in compound libraries).

The present invention encompasses mutant polypeptides that include the polypeptide sequence of a naturally occurring IL-15 having (a) a mutation (e.g., a deletion mutation) of one or more of the first 48 amino acid residues of the precursor protein and (b) a mutation (e.g., a substitution mutation) of one or both of the glutamine (Q) residues in the C-terminal half of the polypeptide. Such IL-15 mutants can be part of a fusion protein, including those that contain a leader sequence and/or a heterologous (i.e., non-IL-15) sequence, such as the Fc region of an IgG molecule. As with IL-15, the leader sequence or heterologous sequence can be mutant with respect to their wild-type counterparts. Mutants of the Fc region, as described herein, are another aspect of the invention. These Fc mutants can be fused or otherwise joined to other polypeptides (e.g., IL-15), regardless of whether the other polypeptide is mutant or wild-type (e.g., the Fc mutants described herein can be fused to a wild-type IL-15 or any other growth factor (e.g., any other interleukin)).

For example, the agent can be an anti-IL-15 or anti-IL-i 5R antibody (of any of the types (e.g., human or humanized) or variants of antibodies known in the art or described further below (e.g., antigen-binding fragments of anti-IL-15 or anti-IL-15R antibodies)). Alternatively, or in addition, the patient can be treated with a polypeptide consisting of a sequence, or including a sequence, that represents a mutant of a naturally occurring IL-15 (e.g., a human IL-15). While such mutants are described further below, we note here that they can include point-mutated IL-15 molecules (e.g., the mutant can include 1-5 (e.g., one or two) substituted amino acid residues), and any of the IL-15 mutants can be joined to a heterologous polypeptide. The heterologous polypeptide is not an IL-15 (e.g., a wild type or mutant human IL-15) polypeptide, and the fusion proteins of the invention specifically exclude any “fusions” where two portions of an IL-15 molecule are joined to generate a naturally occurring IL-15 molecule). The heterologous portion of the polypeptide may increase the circulating half-life of an IL-15 molecule (e.g., a mutant IL-15 molecule) to which it is joined beyond that of the IL-15 polypeptide alone. More specifically, the heterologous polypeptide can be an immunoglobulin or a portion thereof (e.g., an Fc region of an immunoglobulin) or an albumin or a portion thereof. The portions can vary in size but, when included for the purpose of increasing circulating half-life, must be large enough to achieve that purpose (i.e., the heterologous polypeptide must be large enough to increase the circulating half-life of the IL-15 or IL-15R antagonist to which they are fused).

The IL-15 molecules (e.g., the mutant IL-15 molecules described herein, alone or fused to a heterologous polypeptide) can be chemically modified by conjugation to a water-soluble polymer such as polyethylene glycol (PEG), e.g., to increase stability or circulating half-life.

Mutants of the heterologous polypeptides other than deletion mutants (e.g., fragments) can also be used. Where an Fc region is included, it may contain one or more mutations that may or may not affect its function. Native activity may not be necessary or desired in all cases. In specific embodiments, the agent can be a mutant IL-15/Fcγ2a fusion protein. Any of the mutant IL-15 polypeptides described herein can also be joined to (e.g., fused by way of a peptide bond) an Fc region of IgG (e.g., the Fc region of human IgG1) or a variant thereof. An example is described below in which the IL-15 portion of the agent contains two point mutations and the Fc region contains one. Mutant IL-15 polypeptides can be fused to an Fc region of any type or subtype (e.g., type 1, 2b, 2c, 3, or 4). Similarly, a mutant IL-15 or other polypeptide antagonist can be fused to a human Fc region of any immunoglobulin (e.g., an Fc region from an IgG type 1, 2, 3, or 4, or an Fc region of an IgE, IgA, or IgM).

The Fc region, when present, can be lytic or non-lytic (these forms are described further below), and the heterologous polypeptide can be, or can include, other cytotoxic polypeptides. Any of the agents described herein, whether containing an IL-15 or IL-15R antagonist alone or joined to a heterologous polypeptide that is lytic or non-lytic, can also include a substance that serves as a marker or tag (e.g., a polypeptide (e.g., an epitope tag or fluorescent protein) or radioisotope). Moreover, any of the protein-based antagonists (e.g., any of the mutant IL-15 polypeptides) can include a signal peptide that may be subsequently cleaved from the mature form of the antagonist. Examples of suitable signal peptides are provided below. Such peptides are also referred to in the art as signal sequences or leader sequences.

Unless a different or particular meaning is evident from the context, we use the terms “agent” and “antagonist” interchangeably, and we apply such terms regardless of the entity's nature (e.g., whether the agent or antagonist is a nucleic acid, polypeptide, or chemical compound) or precise configuration (e.g., whether polypeptide agents or antagonists consist only of a mutant IL-15 or whether the mutant IL-15 is joined to (e.g., fused to) one or more heterologous polypeptides).

When used in the context of transplantation (e.g., when administered to a patient who has received, or who is scheduled to receive, a heart, lung, or heart-lung transplant), the IL-15 or IL-15R antagonist will have physical attributes that allow it to improve the patient's status or prognosis following receipt of the transplant. For example, the IL-15 or IL-15R antagonist may prolong the time transplanted tissue survives within the patient (e.g., the sequence of the mutant IL-15 polypeptide may be such that its administration prolongs graft survival) and/or that improves the function of the graft during at least some of the time it is implanted in the patient (e.g., the sequence of the mutant IL-15 polypeptide may be such that a transplanted organ (e.g., a heart) is expected to function more effectively following transplantation than an untreated organ of the same type (e.g., an untreated heart) would be expected to function). In the context of transplantation and in other circumstances (e.g., when used in a research study, clinical trial, or a clinical setting other than transplantation to suppress an IL-15-dependent immune response), the IL-15 or IL-15R antagonist (e.g., a mutant IL-15 polypeptide) will inhibit one or more of the activities exhibited by wild type IL-15 in vivo or in vitro (e.g., in cell or tissue culture).

As noted above, the antagonists described herein can be used to treat patients who have received, or who are scheduled to receive, a heart, lung, or heart-lung transplant. The antagonists can also be used to treat patients who have received, or who are scheduled to receive, a transplant of another organ, tissue (e.g., bone marrow), or cell (e.g., a stem cell or stem cell-containing tissue or preparation), patients who have an autoimmune disease, and patients who have suffered a vascular injury (whether caused by disease, trauma, or a surgical procedure). The vascular injury may present as a Shwartzman reaction, where local or systemic vasculitis is caused by a two-stage reaction. A first encounter with endotoxin can produce intravascular fibrin thrombi. The clearance of these thrombi results in reticuloendothelial blockade, which prevents the clearance of thrombi caused by a second encounter with endotoxin. The encounter may also be one with polyanions, glycogen, or antigen/antibody complexes. The result typically includes tissue necrosis and/or hemorrhage. In pregnancy, gram-negative septicemia during delivery or abortion may serve as the first or provocative encounter.

In specific embodiments, a chimeric polypeptide that includes, or that consists of, a mature human IL-15 polypeptide having point mutations at positions 101 and/or 108 (e.g., Q101D and Q108D; as shown in the mature IL-15 of FIG. 3) and an Fc region having a point mutation at position 119 (e.g., C19A; shown in the heterologous Fc molecule of FIG. 3) can be administered to a patient who has received, or who is scheduled to receive, a transplant; a patient who has been diagnosed as having, or who is at risk for developing, an autoimmune disease; and/or to a patient who has received, or who is at risk for developing, a vascular injury. Regardless of the underlying cause of the immune response, the methods can include the step of identifying the patient in need of treatment.

Where the antagonist includes, or consists of, a mutant IL-15 polypeptide, the mutant IL-15 polypeptide can be expressed by (and subsequently purified from) CHO (Chinese hamster ovary) cells or it can be produced by other cells or processes that generate a polypeptide having the same, or substantially the same, glycosylation pattern as that of a mutant IL-15 polypeptide produced in CHO cells. Similarly, where the antagonist includes, or consists of, a chimeric polypeptide including a mutant IL-15 polypeptide and an Fc region of an immunoglobulin (e.g., the chimeric polypeptide shown in FIG. 3), the chimeric polypeptide can be expressed by (and subsequently purified from) CHO cells. Alternatively, the chimeric polypeptide can be produced by other cells or processes that generate a chimeric polypeptide having the same, or substantially the same, glycosylation pattern as that of a corresponding polypeptide produced in CHO cells.

The agents within the invention are not limited to IL-15 or IL-15R antagonists (e.g., a mutant IL-15 polypeptide described herein) that block activity to any certain degree; a useful agent is one that blocks IL-15-mediated signal transduction to any beneficial extent in a cell, cell culture, organ, tissue, graft, or patient to which (or to whom) it is administered. Nevertheless, inhibition can be measured in various assays, and an agent within the invention can be characterized as one that blocks activity to a particular extent. For example, an IL-15 or IL-15R antagonist can block about or at least 40% (e.g., 40, 50, 60, 70, 80, 90, 95, or 99%) of one of the actions, measurable in vivo or in vitro, carried out by wild type IL-15. The ability of a mutant IL-15 polypeptide to inhibit wild type IL-15 activity can be assessed in any one or more of the assays known in the art, including any of those that measure receptor binding and/or signal transduction. Activity can also be measured in cell proliferation assays such as the BAF-BO3 cell proliferation assay described in, for example, U.S. Pat. No. 6,451,308.

Where the inhibitor is a protein-based therapeutic agent (e.g., a mutant IL-15 or mutant IL-15-containing protein), the invention also features nucleic acids that encode those agents, vectors (e.g., expression vectors) that include such nucleic acids, host cells containing the vectors (e.g., eukaryotic cells such as CHO cells or COS cells, or prokaryotic cells such as E. coli cells), and methods of making the desired protein-based therapeutic by providing host cells that express the encoded protein (e.g., a mutant IL-15 polypeptide as described herein or a polypeptide in which it is contained). For example, cells that include a nucleic acid encoding a polypeptide described herein can be expanded in tissue culture (e.g., maintained in a liquid culture in which the cells can survive and may proliferate) under conditions that permit protein expression, and the desired protein can be purified from the cells by methods known in the art. For example, a polypeptide antagonist described herein can be purified by column chromatography. Purification can be facilitated by the inclusion of an affinity tag. Regardless of the methods employed, the desired antagonist can be purified to an extent suitable for inclusion in a pharmaceutical composition, and such compositions are also within the scope of the present invention.

The nucleic acids and expression vectors of the invention can include sequences that may facilitate expression and/or direct secretion of the expressed protein. For example, the nucleic acids or vectors can include a promoter and/or enhancer that is associated with a wild type IL-15 gene or that of another gene (e.g., a constitutively active or tissue-specific promoter). Alternatively, or in addition, the nucleic acids and vectors can include a sequence encoding a signal peptide. For example, the nucleic acids and vectors can include an IL-15 signal peptide or that of another interleukin. For example, one could incorporate a nucleic acid sequence encoding a signal peptide naturally associated with IL-1 (e.g., IL-1α or IL-1β), IL-2 (as described in Bamford et al., J. Immunol. 160:4418, 1998) IL-4, or IL-10. Other suitable signal peptides include those of a CD5 (see FIG. 3), CTLA4, or TNF (Tumor Necrosis Factor. The nucleic acids and vectors can also include a polyadenylation signal. Any of the nucleic acid molecules and expression vectors can also lack a polyadenylation signal.

The encoded signal peptide can be, or can include, the sequence MVLGTIDLCSCFSAGLPKTEA (SEQ ID NO:__) or MRISKPHLRSISIQCYLCLLLNSHFLTEAGIHVFILGCFSAGLPKTEA (amino acid residues 1-148 of SEQ ID NO:2) (see Onu et al., J Immunol. 158:255-262, 1997).

The sequence(s) that facilitate expression or direct secretion of a polypeptide can be wild type sequences (e.g., wild type mammalian (e.g., human) sequence(s)), or they can be truncated or otherwise mutated. For example, the signal peptide may be as found in nature or may be truncated or otherwise mutated; what is required is that enough of the wildtype sequence is retained to allow the leader to function (e.g., to direct secretion or otherwise affect the position of the mature protein to which it was attached within the cell). The nucleic acid molecules and vectors can also include sequences encoding one or more selectable markers, such as a sequence encoding a protein that confers antibiotic resistance (e.g., resistance to G418 (conferred by the neomycin-resistance gene neo^{r})), or a marker or tag.

As noted above, the expressed protein can be purified from host cells using purification methods known in the art (for example, protein can be purified from culture supernatants or cell lysates by protein A Sepharose™ affinity chromatography followed by dialysis against PBS and, optionally, filter sterilization). As noted, CHO cells are among those suitable as host cells, and the invention encompasses antagonists produced by transfected CHO cells or cells that produce polypeptides with the same or substantially the same glycosylation pattern as CHO cells. Due to their length, we expect protein therapeutics to be obtained by recombinant methods, but chemical synthesis is also possible.

In addition to compositions such as those described above, the invention further features compositions and methods of improving a patient's status or prognosis following transplantation (e.g., graft function or survival) or in the event of an autoimmune disease, vascular injury, or other event associated with an IL-15-dependent immune response by administering one or more types of IL-15 or IL-15R antagonists and an agent that inhibits CD40L (also known as CD154). The agent that inhibits CD40L can be, e.g., an anti-CD154 antibody or an antigen-binding fragment thereof; a soluble monomeric CD40L, an inhibitory nucleic acid such as an antisense RNA molecule or siRNA that specifically binds a nucleic acid sequence encoding CD40L or a small molecule (e.g., a small organic molecule). Accordingly, pharmaceutical compositions that include an IL-15 or IL-15R antagonist and an agent that inhibits CD40L are within the scope of the present invention, as are kits that include these compositions, in the same or separate containers, and methods of using them. Other combination therapies within the invention include administration of a combination of one or more antagonists of IL-15 or IL-15R. For example, one can administer a mutant IL-15 polypeptide as described herein and an antibody that binds IL-15 or an IL-15R and inhibits signal transduction. Such antibodies are known in the art and are available from the American Type Culture Collection (ATCC, Rockville, Md. (USA)).

The improvement observed in the patient can be any clinically beneficial improvement or reduction of risk (e.g., risk of rejection or impaired graft function). For example, where the patient is a transplant recipient, the treatment can improve the way in which the transplanted organ or tissue functions and/or the length of time it survives in the patient (function and survival can be relative to the degree of function or length of survival one would expect for a transplant that is untreated with an agent or method of the invention but otherwise comparable). Following transplantation, or where the patient has an autoimmune disease or vascular injury, the treatment can improve an objective sign or subjective symptom of the disease or injury.

Where the methods include administration of two active agents (e.g., an IL-15/IL-15R antagonist(s) and the CD40L inhibitor), they may be administered together. For example, the agents can be administered at the same time or by the same route in separate dosage forms or a single dosage form. Alternatively, the agents can be administered separately (e.g., at different times in the course of the treatment regime and/or by different routes). As our methods encompass simultaneous administration, the invention also features compositions (e.g., pharmaceutically acceptable compositions) in which two types of agents (i.e., the IL-15/IL-15R antagonist and the CD40L inhibitor) are mixed or otherwise combined. Moreover, the two types of agents (whether combined or within separate containers) can be assembled as a kit, as can an IL-15 or IL-15R antagonist alone. Kits containing these agents, instructions for their use (which may be printed or conveyed in another medium (e.g., by audible or audiovisual signals), and, optionally, paraphernalia required for administration to a patient (including one or more of a needle, syringe, alcohol swabs, tubing, cannulas, bandages, and the like) are within the scope of the present invention. The composition(s) can be administered to patients in accordance with dosing regimes perfected by those of ordinary skill in the art and/or in a manner consistent with the schedules shown to be effective in our animal studies (see below).

The agents of the present invention, depending upon their precise nature, may have one or more desirable attributes (e.g., a characteristic that can be advantageously exploited in a treatment regime). For example, because an IL-15R antagonist can differ from wild type IL-15 by as few as one or two substituted residues, the antagonists are unlikely to elicit an undesirable immune response. Antagonists that include IL-15 mutants that bind their receptor with the same, or substantially the same, high affinity as wild type IL-15 can compete effectively with wild type IL-15 for receptor binding (any of the mutant IL-15-containing polypeptides described herein can be analyzed in competitive receptor binding assays). Further, as noted above, IL-15 mutants can be modified to remain active in the circulation for a prolonged period of time. Due to these attributes, methods of treatment with IL-15 or IL-15R antagonists may be superior to methods of treatment that rely on antibodies or toxins to modulate the immune response. Other features and advantages of the invention will be apparent from the accompanying drawings and description, and from the claims.

Although methods and materials similar or equivalent to those described herein can be used in the practice or testing of the present invention, useful methods and materials are described below. The materials, methods, and examples are illustrative only and not intended to be limiting.

### BRIEF DESCRIPTION OF THE DRAWINGS

FIG. 1 is the wild type IL-15 nucleic acid and predicted amino acid sequence, including a signal sequence (SEQ ID Nos. 1 and 2, respectively).

FIG. 2 is the mutant IL-15 nucleic acid and predicted amino acid sequence, including a signal sequence. The wild type codon encoding glutamine at position 169, CAG, and the wild type codon encoding glutamine at position 176, CAA, have both been changed to GAC, which encodes aspartate (SEQ ID Nos. 3 and 4, respectively).

FIG. 3 is a representation of the sequence of a human mutant IL-15 fused to a human IgG1 Fc molecule. A leader sequence is also shown (represented by negative numbers and misaligned (SEQ ID NO:5). The mutant IL-15 sequence is numbered in the figure as residues 1-114. The sequence numbered in the figure as residues 115-346 is an Fc region including the hinge and segments C2 and C3. The fused mutant IL-15 sequence and the Fc region are represented by SEQ ID NO:6. Glycosylation sites are underlined and point mutations are highlighted with arrows.

FIG. 4 is a Table showing the results of treating murine transplant recipients with an IL-15/Fc fusion protein, as described in the Examples.

FIG. 5 is a set of graphs comparing -levels of expression of cytotoxic T cell markers, inflammatory markers, and cytokines in heart allografts from mice treated i.p. with an antagonistic IL-15 mutant/Fcγ2a fusion protein, CRB-15 (T) or treated with control IgG2a (C). Expression levels of the following genes detected in grafted hearts removed 5 days after transplantation are shown: Fas Ligand (FasL), Perforin (Perf), Granzyme B (GmB), interleukin-1β (IL-1β), tumor necrosis factor-a (TNF-α), interferon-g (IFN-γ), and interleukin-4 (IL-4).

FIG. 6 is a graph showing survival of heart allografts in mice treated with IgG2a, CRB-15, or a non-lytic form of CRB-15, CRB-15 nl.

FIG. 7 is a graph showing survival of heart allografts in mice treated with IgG2a, CRB-15, anti-CD 154, or CRB-15 and anti-CD 154.

FIG. 8A is a graph showing survival of minor histocompatibility-mismatched heart allografts in mice treated with a short course of either IgG2a or CRB-15.

FIG. 8B is a graph showing survival of secondary heterotopic cervical heart allografts in mice treated as described for FIG. 8A during the initial transplant, without any further immunosuppressive treatment during the secondary transplant. The secondary allograft was implanted more than 100 days after survival of the primary transplant.

FIG. 9A is a graph showing survival of pancreatic islet allografts in fully MHC-mismatched control or CRB-15-treated animals.

FIG. 9B is a graph showing survival of secondary islet allografts from Balb/c (H-2d) or B10.A (H-2d) donors without further immunosuppression.

### DETAILED DESCRIPTION

The compositions of the present invention include agents that inhibit one or more of the actions of wild type IL-15. While the agents of the invention are not limited to those that act by any particular mechanism, we note here that they may antagonize IL-15 by inhibiting the expression or activity of wild type IL-15 or the IL-15R, they may bind IL-15 or the IL-15R and inhibit signal transduction, or they may inhibit signal transduction downstream from receptor binding. The inhibition can occur before or during an immune response, which may be provoked by the receipt of non-self cells or in the course of an autoimmune disease, as the agents preferably selectively inhibit the activity of cells that naturally bind wild type IL-15. The agents, by virtue of inclusion of a lytic or toxic component, can also be used to kill the cells to which they bind (e.g., cells expressing an IL-15 receptor). Mutant IL-15 polypeptides, proteins or protein complexes containing them (e.g., fusion proteins or covalently or non-covalently bound protein complexes), and other agents of the invention are described in more detail below, as are methods in which these agents can be made and used.

Polypeptide agents. As noted, methods of treating a patient who is experiencing, or who may soon experience, an unwanted immune response in which IL-15 participates (e.g., a transplant recipient, a patient who has an autoimmune disease or a vascular injury) can be carried out using one or more polypeptides that are, or that include, mutants of wild type IL-15. Functionally, such polypeptides antagonize wild type IL-15 or its receptor and, when administered to the patients described herein, do so to a clinically beneficial extent. While the invention is not limited to agents that work by any particular mechanism, we believe these polypeptides can antagonize wild type IL-15 by binding to or otherwise interacting with the IL-15R in a way that inhibits signal transduction.

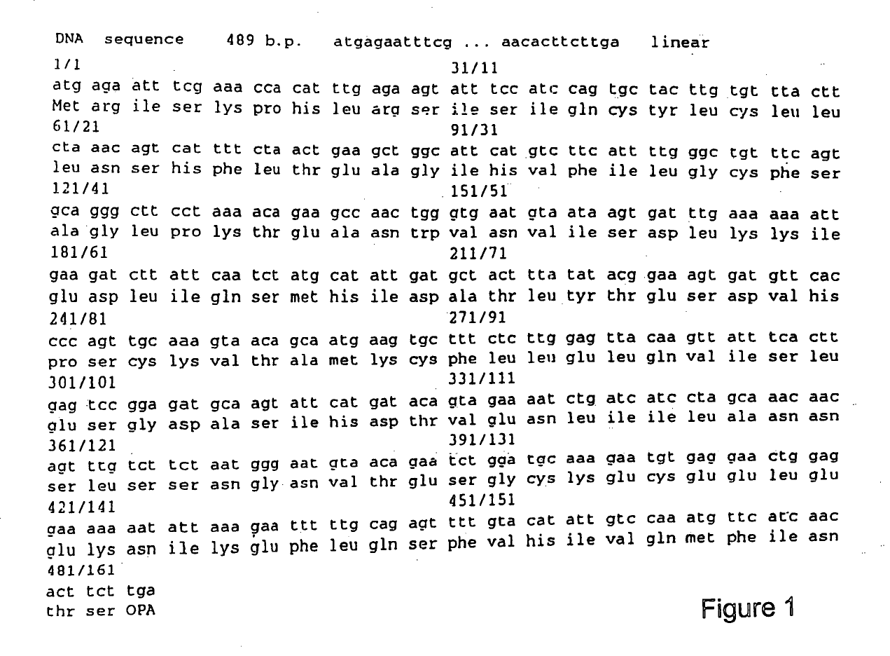

With respect to the sequence of the mutant IL-15, in various embodiments, such polypeptides will be at least or about 65% (e.g., at least or about 63, 64, 65, 66, or 67%) identical to a wild type IL-15; at least or about 75% (e.g., at least or about 73, 74, 75, 76, or 77%) identical to a wild type IL-15; at least or about 85% (e.g., 83, 84, 85, 86, or 87%) identical to a wild type IL-15;, or at least or about 90% (e.g., 89, 90, 91, 92, 93, 94, 95, 96, 97, 98, or 99%) identical to a wild type IL-15. The mutant and wild type polypeptides compared can be of the same species. For example, the wild type IL-15 can be a human IL-15, and one can introduce mutations into the human sequence to produce a mutant IL-15. The wild type sequence may be referred to as the reference standard. Moreover, the referenced wild type sequence and the mutant to which it is compared can constitute a mature form of an IL-15 (e.g., amino acid residues 49-162 of FIG. 1) or a precursor that includes a signal peptide (e.g., amino acid residues 1-48 of FIG. 1). More specifically, the wild type sequence and the mutant to which it is compared can constitute a form of IL-15 that includes the signal peptide MVLGTIDLCSCFSAGLPKTEA (SEQ ID NO:26) followed by amino acid residues 49-162 of FIG. 1. The mutant IL-15 polypeptides can: (a) include a mutation at position 149 of SEQ ID NO:2, (b) exhibit at least 90% identity to a corresponding wild type IL-15, and (c) inhibit one or more of the activities mediated by wild type IL-15.

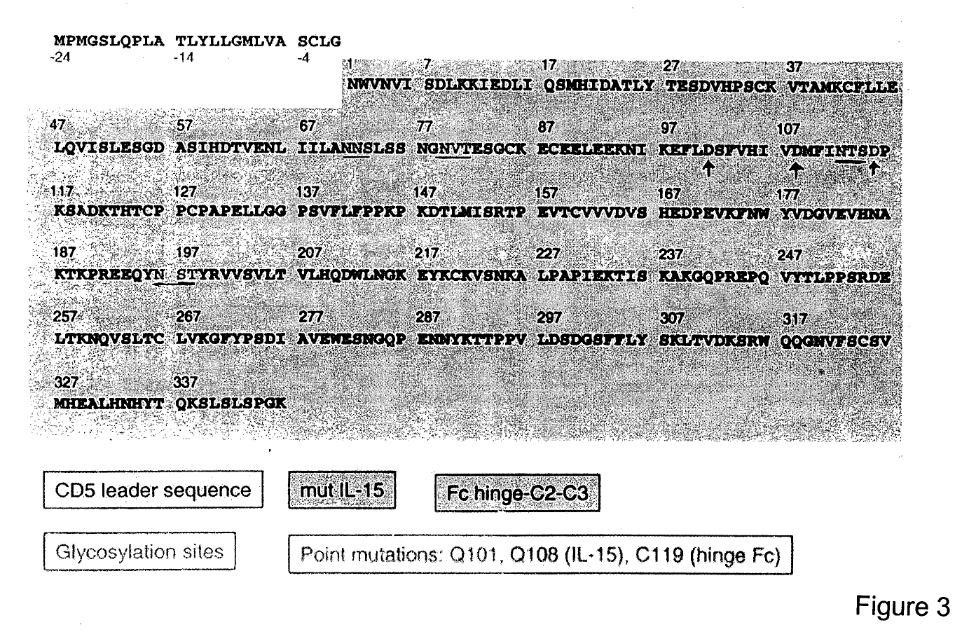

A wild type IL-15 polypeptide that is joined to (e.g., fused to) a heterologous polypeptide can also serve as a reference standard for a corresponding protein. For example, a wild type IL-15 polypeptide fused to a wild type Fc region of an immunoglobulin can serve as the reference standard for a mutant IL-15 polypeptide fused to a mutant or wild type Fc region of an immunoglobulin. Such agents can exhibit the same certain degrees of identity to a corresponding reference standard as set forth above with respect to IL-15 alone. For example, where the agent includes a mutant IL-15 and an Fc region, the mutant IL-15 and Fc region can be at least or about 90% (e.g., 89, 90, 91, 92, 93, 94, 95, 96, 97, 98, or 99%) identical to a reference standard consisting of a corresponding wild type IL-15 joined, in the same manner and orientation as the mutant IL-15, to a wild type Fc region. The mutation(s) in the antagonist can be within the Fc region as well as within the IL-15 polypeptide. For example, as shown in FIG. 3, the Fc region can include a mutation of the first glutamine residue and the first cysteine residue (in FIG. 3, the sequence EPKSCD (SEQ ID NO:27) is mutated to DPKSAD (SEQ ID NO:28). In the antagonists described herein, the Fc region can be a human Fcγ1 domain having either or both of these mutations. Antagonists that include, or that consist of, a mutant IL-15 polypeptide and an Fc region can: (a) include a mutation at position 101 and/or position 108 of SEQ ID NO:6 and a mutation within the Fc region (e.g., a mutation at position 115 and/or 119 of SEQ ID NO:6), (b) exhibit at least 90% identity to a corresponding polypeptide that includes, or that consists of, the corresponding wild type IL-15 and Fc regions, and (c) inhibit one or more of the activities mediated by wild type IL-15 (e.g., signal transduction through the IL-15R).

In one embodiment, the Fc region is a mutated human IgG1 Fc region comprising, or consisting of, the following sequence:

(SEQ ID NO:_)

DPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

The percentage of identity between a subject'sequence and a reference standard can be determined by submitting the two sequences to a computer analysis with any parameters affecting the outcome of the alignment set to the default position. In some instances (e.g., where any mutations are point mutations), a subject sequence and the reference standard can exhibit the required percent identity without the introduction of gaps into one or both sequences. In many instances, the extent of identity will be evident without computer assistance. For example, one of ordinary skill in the art would readily be able to conclude that introducing an addition, deletion, or substitution of a single amino acid residue into SEQ ID NO:2 would produce a mutant IL-15 polypeptide that is at least 99% identical to SEQ ID NO:2; that introducing an addition, deletion, or substitution of two amino acid residues (or any combination of two such mutations) into SEQ ID NO:2 would produce a mutant IL-1 polypeptide that is at least 98% identical to SEQ ID NO:2; and so forth.

As illustrated by the statements above, the mutant IL-15 can differ from a corresponding wild type IL-15 (e.g., a mutant human IL-15 can differ from a wild type human IL-15) by one or more deletions, insertions, or amino acid substitutions, whether the substitutions represent conservative or non-conservative amino acid substitutions, in any part or region of the polypeptide, including the carboxy-terminal domain, which is believed to bind the IL-2Rα subunit (e.g., residues 44-52 of SEQ ID NO:6 (LLELQVISL (SEQ ID NO:7)) or residues 64-68 of SEQ ID NO:6 (ENLII) (SEQ ID NO:8); see Bernard et al., J. Biol. Chem. 279:24313-24322, 2004). One or more mutations can also be introduced within the IL-2Rγ binding domain or the IL-2Rβ binding domain. As noted above, the mutant polypeptides described herein that include all or part of an Fc region are polypeptides of the invention, even when not fused or otherwise joined to another polypeptide or when fused or otherwise joined to another polypeptide such as IL-15 or another therapeutic polypeptide, whether mutant or wild type.

Regardless of the number or position of a mutation or the polypeptide within which it is contained (e.g., whether within IL-15 or a heterologous polypeptide such as an Fc region of an IgG), the amino acid residue that is added (to create, for example, an addition or substitution mutant) can be naturally occurring or non-naturally occurring.

More specifically, the mutant IL-15 polypeptide can differ from wild type IL-15 by a mutation (e.g., a substitution) of residue 149 or 156 (of SEQ ID NO:2) when fused to, for example, a mutant Fc region, or by a mutation (e.g., a substitution) of both residues 149 and 156, whether or not an Fc region is included. The antagonist can, in addition, include one or more deletions, insertions, or amino acid substitutions of (or within) the residues of SEQ ID NO:7 or SEQ ID NO:8. For example, the antagonist can include a mutation at residue 149, residue 156, or both (of SEQ ID NO:2) and a mutation at one or more of residues 1 12, 113, and 116 (of SEQ ID NO:2).

Where the mutant contains a substitution mutation, the substitution can be such that the mutant IL-15 polypeptide differs from wild type IL-15 by the substitution of aspartate for glutamine at residue 149, at residue 156, or both. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine, and such substitutions can be incorporated in the mutant IL-15 polypeptides described herein. In a specific embodiment, a mutant of human IL-15 is fused to a wild-type or mutant human IgG1 Fc region. This human IL-15/Fc chimera or any of the IL-15-containing antagonists described herein may be optionally linked to a CD5 leader sequence, as shown in FIG. 3 (i.e., a CD5 leader sequence having the following residues: MPMGSLQPLATLYLLGMLVASCLG (SEQ ID NO:__).

We use the terms “protein,” “polypeptide,” and “peptide” interchangeably to refer to any chain of three or more amino acid residues, which may be joined by peptide bonds. For example, we may refer to IL-15, whether wild type or mutant, as an IL-15 protein, polypeptide, or peptide. In the polypeptide-containing agents of the invention, one or more of the residues may be post-translationally modified (e.g., glycosylated or phosphorylated). As noted, and for example, an IL-15 antagonist can be glycosylated as CHO cells glycosylate a mutant IL-15, such as the mutant IL-15-containing polypeptide shown in FIG. 3 (e.g., the sites underlined in FIG. 3 (NNS site at residues 71-73; NVT site at residues 80-82; NTS site at residues 112-114; and NST site at residues 196-198) can be glycosylated).

As noted, where the agent is a polypeptide, it can be a chimeric polypeptide that includes a mutant IL-15 polypeptide and a heterologous polypeptide that confers some benefit on the IL-15 portion of the polypeptide. For example, the heterologous polypeptide may increase the circulating half-life of the mutant IL-15 in vivo. We use the term “circulating half-life,” as it is used in the art, to mean the period of time that elapses before a given amount of a substance that is present in the circulatory system of a living animal (e.g., a human patient) is reduced by one half. The heterologous polypeptide can be a serum albumin, such as human serum albumin, or a portion thereof, or it may include all, or part of, the Fc region of an immunoglobulin (i.e., any or all of an immunoglobulin lacking, in its entirety, the variable region of a heavy or light chain). The hinge region of the immunoglobulin may be included.

Where the antagonist employed includes (e.g., is fused to) an Fc region, that region may be altered (e.g., by inclusion of a mutation) to convey a desirable characteristic on the fusion protein or, more specifically, on the IL-15 or IL-15R portion of the molecule. For example, one can mutate the FcR binding and C1q-binding domains of the Fc fragment to render the Fc unable to direct antibody-dependent cell-mediated cytotoxicity and complement-dependent cytotoxic activities. For example, the Fc regions of human immunoglobulins (e.g., the human Fcγ1 isotype) are able to bind effectively to cells expressing high affinity receptors (e.g., an FcγR1 receptor) and possess a complement (C1q) binding domain, and thus are able to facilitate Ab-dependent cell-mediated cytotoxicity (ADCC) and complement dependent cytotoxicity (CDC). The complement (C1q) and FcγR1 binding sites of a human Fcγ1 fragment can be mutated by, for example, site-directed mutagenesis as described by Duncan and Winter (Nature 332:738, 1988) and Duncan et al. (Nature 332:563, 1988), respectively. Duncan and Winter used a surface scanning technique, which systematically removes the side chains from amino acid residues, to localize the C1q binding site within the CH₂ domain. The subject sequence was of a mouse IgG2b isotype, and C1q binding was localized to Glu318, Lys320, and Lys322. These residues are relatively conserved in other antibody isotypes (Duncan and Winter report that residues Glu318, Lys320, and Lys322 are conserved in all the human IgGs), and a peptide mimic of this sequence was able to inhibit complement lysis. Accordingly, the antagonists described herein can include Fc regions having mutations at one or more of these three positions. For example, any or all of Glu318, Lys320, and Lys322 can be substituted with another amino acid residue such as alanine. For further information regarding C1q, one can consult Duncan and Winter (Nature 332:738, 1988) and for additional information regarding FcγR1, Duncan et al. (Nature 332:563, 1988). Alternatively, the Fc region can be lytic (i.e., able to bind complement or to lyse cells via another mechanism, such as antibody-dependent complement lysis (ADCC; see U.S. Pat. No. 6,410,008)). Fc regions that are considered lytic can be wild type; can contain a mutation that does not affect their ability to lyse cells (e.g., cells in vivo); or can include a mutation that enhances their ability to lyse cells.

In other instances, the chimeric polypeptide (or “fusion” protein) may include an IL-15 or IL15R antagonist (e.g., a mutant IL-15) and a polypeptide that functions as an antigenic tag, such as the FLAG sequence. FLAG sequences are recognized by biotinylated, highly specific, anti-FLAG antibodies (see, U.S. Pat. No. 6,451,308; see also Blanar et al., Science 256:1014, 1992; LeClair et al., Proc. Natl. Acad. Sci. USA 89:8145, 1992).

In some embodiments, a mutant IL-15 polypeptide is conjugated to a water-soluble polymer, e.g., to increase stability or circulating half life or reduce immunogenicity. Clinically acceptable, water-soluble polymers include, but are not limited to, polyethylene glycol (PEG), polyethylene glycol propionaldehyde, carboxymethylcellulose, dextran, polyvinyl alcohol (PVA), polyvinylpyrrolidone (PVP), polypropylene glycol homopolymers (PPG), polyoxyethylated polyols (POG) (e.g., glycerol) and other polyoxyethylated polyols, polyoxyethylated sorbitol, or polyoxyethylated glucose, and other carbohydrate polymers. Methods for conjugating polypeptides to water-soluble polymers such as PEG are described, e.g., in U.S. patent Pub. No. 20050106148 and references cited therein.

The polypeptides of the invention (e.g., a mutant IL-15/Fc polypeptide described herein) can be dimerized, and such dimers as well as methods in which they are administered to a patient are within the scope of the present invention. The dimer can consist of two identical polypeptides (e.g., two copies of the polypeptide represented by SEQ ID NO:7) or two non-identical polypeptides (one of which can be the polypeptide represented by SEQ ID NO:7). Regardless of the precise polypeptides used, the C-termini and N-termini can be aligned or roughly aligned. For example, where each of the polypeptides includes an Fc region at the N-terminus, the dimer can include molecular bonds between the two Fc regions (e.g., disulfide bonds between one or more of the cysteine residues within one Fc region and the other).

A mutant IL-15 polypeptide, whether alone or as a part of a chimeric polypeptide or other protein complex, can be encoded by a nucleic acid molecule, including a molecule of genomic DNA, cDNA, or synthetic DNA. Any desired mutation can be introduced into a corresponding wild type IL-15 gene sequence by molecular biology techniques well known in the art. Just as the mutant IL-15-containing polypeptides can be described as having a certain “percent identity” with a corresponding wild type protein (a reference standard), the nucleic acid molecules encoding them can be described as having a certain “percent identity” with a corresponding wild type nucleic acid sequence. The nucleic acid molecules can also be characterized in terms of the polypeptides they encode. For example, a nucleic acid molecule within the scope of the present invention can encode a polypeptide that exhibits a certain minimal amount of identity to a reference polypeptide. For example, a nucleic acid molecule can encode a polypeptide that is at least or about 90% (e.g., 89, 90, 91, 92, 93, 94, 95, 96, 97, 98, or 99%) identical to a reference standard consisting of a corresponding polypeptide (e.g., a wild type IL-15 polypeptide).

As the nucleic acid molecules and encoded mutant polypeptides are not naturally occurring, it is unnecessary to refer to them as being isolated or purified solely for the purpose of distinguishing them from an article undisturbed in nature. When the nucleic acid molecules, vectors containing them, and/or encoded polypeptides are used (for example, for one of the purposes described herein), they may be isolated from other biological materials to the extent necessary or desired. For example, when included within a composition (e.g., a composition for use in an assay; a composition for administration to a cell in cell or tissue culture; or a composition for administration to a patient), the nucleic acid, vector, or polypeptide can be substantially isolated or purified. For example, the nucleic acid, vector, or polypeptide can be free from at least 50% (e.g., at least 50, 60, 70, 80, 90, 95, 98, or 99%) of the biological material with which it was formerly associated. For example, a mutant IL-15-containing polypeptide can be at least 98% free from the material of the cell in which it was expressed.

As described further below, the nucleic acid molecules and/or vectors can be administered to a patient who has received, or who is scheduled to receive, a transplant (e.g., a heart, lung, or heart-lung transplant) or who is, or who may soon, suffer from an immune response in which IL-15 is expressed (e.g., an immune response that occurs in the context of an autoimmune disease or a vascular injury). The nucleic acid molecules or vectors can be administered in addition to, or in lieu of, administration of the encoded polypeptide. Similarly, the nucleic acid molecules and/or vectors may be used in any of the combination therapies that include an encoded polypeptide. For example, in addition to, or in lieu of, administering two types of mutant IL-15 polypeptides, one can administer nucleic acid molecules or vectors that encode both types of polypeptides. The nucleic acid molecules or vectors can also be administered in conjunction with other therapeutic agents (e.g., the anti-CD 154 antibodies described above and/or traditional immunosuppressants such as cyclosporin).

The nucleic acid molecules may be contained within a vector that is capable of directing expression of a mutant IL-15 polypeptide in, for example, a cell that has been transduced (e.g., transfected) with the vector. These vectors may be viral vectors, such as retroviral, adenoviral, or adenoviral-associated vectors, as well as plasmids or cosmids. More specifically, the vector can be a modified herpes virus, simian virus 40 (SV40), papilloma virus, or a modified vaccinia Ankara virus.

Suitable vectors include T7-based vectors for use in bacteria (see, e.g., Rosenberg et al., Gene 56:125,1987), the pMSXND expression vector for use in mammalian cells (Lee and Nathans, J. Biol. Chem. 263:3521, 1988), and baculovirus-derived vectors (for example, the expression vector pBacPAK9 from Clontech, Palo Alto, Calif., USA) for use in insect cells. While additional promoters are described elsewhere, we note that a T7 promoter can be used when the host cells are bacterial, and a polyhedron promoter can be used in insect cells.

Mammalian expression vectors typically include nontranscribed regulatory elements such as an origin of replication, a promoter sequence, an enhancer linked to the structural gene, other 5′ or 3′ flanking nontranscribed sequences (e.g., ribosome binding sites, a polyadenylation site, splice donor and acceptor sites, and transcriptional termination sequences). Regulatory sequences derived from Polyoma, Adenovirus 2, Simian Virus 40 (SV40), and human cytomegalovirus are frequently used for recombinant expression in mammalian cells. For example, SV40 origin, early and late promoter, enhancer, splice, and polyadenylation sites may be used to provide the other genetic elements required for expression of an IL-15 mutant DNA sequence in a mammalian host cell. Cytomegalovirus or metallothionein promoters are also frequently used in mammalian cells.

Cells (e.g., eukaryotic cells) that contain and express a nucleic acid molecule encoding any of the mutant IL-15 polypeptides described herein are also features of the invention, and they can be used in methods of making the mutant IL-15 -containing polypeptides described herein or administered to patients receiving a transplant (e.g., a heart transplant, lung transplant, or heart-lung transplant) or otherwise in need of modulating the IL-15-mediated part of an immune response.

Examples of suitable mammalian host cell lines for production of mutant IL-15 polypeptides include: CHO cells; COS cell lines derived from monkey kidney, (e.g., COS-7 cells, ATCC number CRL 1651); L cells; C127 cells; 3T3 cells (ATCC number CCL 163); HeLa cells (ATCC number CCL 2); and BHK (ATCC number CRL 10) cell lines.

Where the cells are administered to patients receiving a transplant, they may be cells within the transplant itself. Other administered cells may be autologous to the patient (e.g., cells such as blood cells, bone marrow cells, or stem cells that are removed from the patient, transduced to express a polypeptide described herein, and readministered). The method of transduction, the choice of expression vector, and the host cell may vary. The precise components of the expression system are not critical. It matters only that the components are compatible with one another, a determination that is well within the ability of one of ordinary skill in the art. Furthermore, for guidance in selecting an expression system, skilled artisans may consult Ausubel et al., Current Protocols in Molecular Biology (1993, John Wiley and Sons, New York, N.Y.), Pouwels et al., Cloning Vectors: A Laboratory Manual (1985, Supp. 1987), and similar teaching manuals.

In one embodiment, a polypeptide described herein (e.g., the polypeptide represented by SEQ ID NO:6) is generated by providing CHO cells transduced (e.g., transfected) with a nucleic acid molecule or vector construct (e.g., a retroviral vector) that expresses the polypeptide; culturing the cells for a time and under conditions sufficient to allow expression of the polypeptide, and purifying the polypeptide from the cells. Polypeptides made by such a method are within the scope of the present invention.

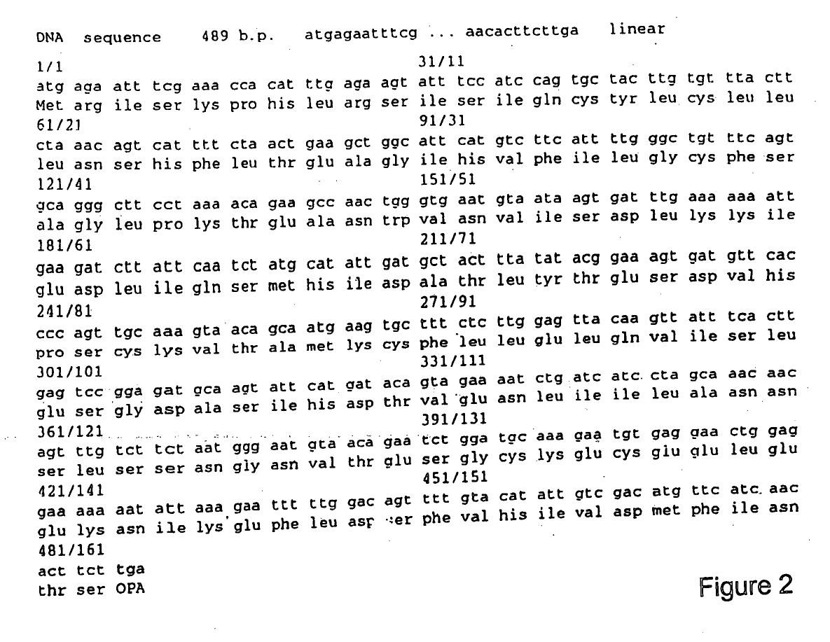

Genetic Construction of a mutant IL-15: in one embodiment, the human IL-15 protein bearing a double mutation (Q149D; Q156D) was designed to target the putative sites critical for binding to the IL-2Rα subunit. The polar, but uncharged glutamate residues at positions 149 and 156 (FIG. 1) were mutated into acidic residues of aspartic acid (FIG. 2) utilizing PCR-assisted mutagenesis. A cDNA encoding the double mutant of IL-15 was amplified by PCR utilizing a synthetic sense oligonucleotide [5′-GGAATTCAACTGGGTGAATGTAATA-3′ (SEQ ID NO.:9); Eco RI site (underlined hexamer) plus bases 145-162] and a synthetic antisense oligonucleotide [5′-CGGGATCCTCAAGAAGTGTTGATGAACATGTCGACAATATGTACAAAACT GTCCAAAAAT-3′(SEQ ID NO.:10); Bam HI site (underlined hexamer) plus bases 438-489; mutated bases are singly underlined. The template was a plasmid containing cDNA that encodes human FLAG-HMK-IL-15. The amplified fragment was digested with Eco RI/Bam HI and cloned into the pAR(DRI)59/60 plasmid digested with Eco RI/Bam HI as described (LeClair et al., Proc. Natl. Acad. Sci. USA 89:8145, 1989). The presence of a mutation at residue 156 was confirmed by digestion with SalI; the mutation introduces a new SalI restriction site. We verified the mutations by DNA sequencing according to standard techniques. Using this same strategy, we prepared mutants that contain only a single amino acid substitution, either at position 149 or at position 156.

The strategy described above, or methods that vary in routine ways from those in that strategy, can be used to incorporate any other amino acid, or any series of amino acids, in place of the glutamate residues at positions 149 or 156 or to introduce amino acid substitutions at one or more positions (e.g., 3, 4, 5, 5-10, or 10-15 positions) other than 149 and/or 156. The strategy described above, or methods that vary in routine ways from those in that strategy, can be used to generate and express mutant IL-15-containing polypeptides that may or may not include a signal peptide; that may or may not include a heterologous polypeptide to alter circulating half-life or carry out effector functions such as ADCC and CDC; or that may or may not contain a selectable or detectable marker or tag. Examples of suitable marker genes include β-lactamase, chloramphenicol acetyltransferase (CAT), adenosine deaminase (ADA), aminoglycoside phosphotransferase (neo4, G418r), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). The “Flag-tag” sequence can also be used. For example, a tagged IL-15-containing molecule can be prepared as described in U.S. Pat. No. 6,451,308 (and can be, for example, the FLAG-HMK-IL-15 chimera described therein).

Pharmaceutical Compositions and Methods of Treatment: By modulating the events mediated by the IL-15 receptor complex, mutant IL-15 polypeptides can modulate the immune response. Accordingly, the nucleic acid molecules, vectors, cells, and polypeptides described herein can be formulated as pharamaceutical compositions and can be administered to patients. The patient can be diagnosed as having, or determined to be at risk for developing an autoimmune disease, including but not limited to the following: (1) a rheumatic disease such as rheumatoid arthritis, systemic lupus erythematosus, Sjogren's syndrome, scleroderma, mixed connective tissue disease, dermatomyositis, polymyositis, Reiter's syndrome or Behcet's disease (2) type II diabetes (3) an autoimmune disease of the thyroid, such as Hashimoto's thyroiditis or Graves' Disease (4) an autoimmune disease of the central nervous system, such as multiple sclerosis, myasthenia gravis, or encephalomyelitis (5) a variety of phemphigus, such as phemphigus vulgaris, phemphigus vegetans, phemphigus foliaceus, Senear-Usher syndrome, or Brazilian phemphigus, (6) psoriasis, and (7) inflammatory bowel disease (e.g., ulcerative colitis or Crohn's Disease). The compositions described herein may also be useful in the treatment of acquired immune deficiency syndrome (AIDS). Other patients amenable to treatment include patients who have received, or who are scheduled to receive, a transplant of biological materials, such as an organ, tissue, or cell transplant. The compositions are useful whenever the the patient and the transplant donor have a complete or partial immunological incompatibility, as occurs to some degree in all instances except an autologous (self-to-self) transplant or a transplant from an identical twin. The transplanted organ, tissue, or cell, can be any organ, tissue, or cell. These include, without limitation, bone, bone marrow, connective tissue (e.g., tendons, cartilage, and ligaments) skin, muscle, adipose cells or tissue including adipose cells, an eye, a heart, a lung, or heart-lung complex, endocrine tissue (e.g., islet or other cells from the pancreas, cells from the thyroid, parathyroid, or adrenal gland), spleen, liver, or kidney. In addition, patients who have received a vascular injury, which may manifest as vasculitis, would benefit from the compositions and methods described herein.

The methods of the invention can be carried out by administering an antagonist (e.g., a mutant IL-15 polypeptide, including those fused to or otherwise joined to a heterologous polypeptide)). The antagonists can be administered alone; two or more types of antagonists can be administered; an antagonist or combination of antagonists can be used in conjunction with an antibody that inhibits CD40L (e.g., an anti-CD154 antibody, or a soluble monomeric CD40L, as described in U.S. Pat. No. 6,264,951); or the antagonist(s) can be administered with other agents used for immune suppression (i.e., the invention includes combination therapies in which the antagonists are administered to a patient).

The mutant IL-15-containing molecules described herein can be used to suppress the immune response in a patient by administering a dose of mutant IL-15 sufficient to competitively bind the IL-15 receptor complex and thereby modulate the immune response. The polypeptide administered may be, or may include, a mutant IL-15 polypeptide, as described herein. As noted above, the method may be used to treat a patient who has received a transplant of biological materials, such as an organ, tissue, or cell transplant. Moreover, the transplant may be of an organ (e.g., heart), tissue, or cell that is partially of fully MHC mismatched. For example, the donor of the transplanted tissue and the transplant recipient may be non-identical twins, siblings, parent and child, or more distantly related (e.g., grandparent and child, cousins, niece or nephew and aunt or uncle, and so forth). The donor and recipient may also be unrelated. Through the administration of a lytic form of the mutant IL-15 polypeptide, it is possible to selectively kill autoreactive or “transplant destructive” immune cells without massive destruction of normal T cells.

In the immunosuppressive and therapeutic applications described above, the polypeptide (e.g., a mutant IL-15) may be administered with a physiologically-acceptable carrier, such as physiological saline by any standard route including a parenteral route (e.g., intraperitoneally, intramuscularly, subcutaneously, or intravenously). While the invention is not so limited, as therapeutic proteins have been administered intravenously, we expect that intravenous administration will provide a convenient route for administration of the polypeptide antagonists of the present invention (e.g., polypeptide antagonists of IL-15 or IL-15R). It is well known in the medical arts that dosages for any one patient depend on many factors, including the general health, sex, weight, body surface area, and age of the patient, as well as the particular compound to be administered, the time and route of administration, and other drugs being administered concurrently. Dosages for the polypeptide of the invention will vary, but a preferred dosage for intravenous administration is approximately 0.01 mg to 100 mg/kg (e.g., 0.01-1 mg/kg). Determination of correct dosage for a given application is well within the abilities of one of ordinary skill in the art of pharmacology. The determined dosage may be given daily or several times daily. The studies described below suggest a regimen where the IL-15 antagonist is administered every second day following receipt of the transplant. The administration can be limited (i.e., it can be given for a number of days or weeks (e.g., 2, 4, 6, or 8 days or weeks) following the transplantation, or unlimited (i.e., it can be given for a substantial period of time, including up to the remaining lifetime of the transplant recipient).

Additional description: In various embodiments, the methods carried out can be methods of suppressing (i.e., lessening, to a perceptible extent) an IL-15-dependent immune response (i.e., an immune response in which IL-15 is produced and contributes to an unwanted activation of the immune system)). The methods can include the steps of providing a patient who has experienced, or who is at risk for experiencing, an IL-15-dependent immune response (e.g., following receipt of a graft from a donor, the graft including an organ or biological tissue); and administering to the patient an amount of a physiologically acceptable composition (e.g., a solution or other pharmaceutical formulation) that includes a polypeptide that includes the sequence represented by SEQ ID NO:6 (see FIG. 3) or a nucleic acid sequence encoding a polypeptide that includes the sequence represented by SEQ ID NO:6. The amount of the composition administered is an amount sufficient to suppress the IL 15-dependent immune response. Thus, where such a response follows the receipt of an organ or other biological tissue from a donor, the composition can prolong the survival of the graft in the patient or improve graft function. The transplant may have originated in a donor having a complete or partial immunological incompatibility with the patient. The transplant can include a wide variety of organs and/or tissue types, including a heart, kidney, skin, liver, or lung. In any of the embodiments described herein, the patient can be a human patient. In any of the embodiments described herein, the methods can include administering to the patient an agent that inhibits CD40L (e.g., an anti-CD 154 antibody). An IL-15-dependent immune response may also be provoked in a patient that has, or is at risk of developing, an autoimmune disease (e.g., any of those known in the art and/or described above (e.g., rheumatoid arthritis)) or a disease characterized by vasculitis, and the compositions described herein can be used to treat or prevent such diseases.

The invention features use of a polypeptide, nucleic acid molecule (or vector containing same) as described herein for suppression of an IL-15-dependent immune response. The polypeptide can be, or can include, the sequence represented by SEQ ID NO:6 and the nucleic acid molecule can be, or can include, a sequence encoding the polypeptide of SEQ ID NO:6. The polypeptide can further include, and the nucleic acid can further encode a signal sequence as described herein (e.g., SEQ ID NO:5). The polypeptides, nucleic acids, and vectors described herein can similarly be used for the preparation of a medicament for, for example, suppression of an IL 15-dependent immune response. The following examples help to illustrate the invention and to provide those of ordinary skill in the art with further information they may find useful in practicing the invention. Antagonists, reagents, methods of use and other information taught by way of the examples can be used in the compositions and methods described above. The invention is not limited, however, to the procedures described below.

### EXAMPLES

In the studies described below, we found that administration of a lytic and antagonistic IL-15 mutant/Fcγ2a fusion protein, CRB-15, prevented rejection and induced antigen-specific tolerance of minor histocompatibility complex-mismatched grafts in a B10.Br to CBA/Ca strain combination, and prolonged the survival of transplanted hearts in fully MHC-mismatched recipients in a Balb/c to C57BL/6 mouse strain combination. Prolonged graft survival was accompanied by reduced mononuclear cell infiltration and inflammatory cytokine expression in the treated graft recipients.

Generation of mIL-15/Fc chimeric proteins: cDNA for Fcγ2a can be generated from mRNA extracted from an IgG2a secreting hybridoma using standard techniques with reverse transcriptase (MMLV-RT; Gibco-BRL, Grand Island, N.Y.) and a synthetic oligo-dT (12-18) oligonucleotide (Gibco BRL). The mutant IL-15 cDNA can be amplified from a plasmid template by PCR using IL-15-specific synthetic oligonucleotides. For example, the 5′ oligonucleotide is designed to insert a unique NotI restriction site 40 nucleotides 5′ to the translational start codon, while the 3′ oligonucleotide eliminates the termination codon and modifies the C-terminal Ser residue codon usage from AGC to TCG to accommodate the creation of a unique BamHI site at the mutant IL-15/Fc junction. Synthetic oligonucleotides used for the amplification of the Fcγ2a domain cDNA change the first codon of the hinge from Glu to Asp in order to create a unique BamHI site spanning the first codon of the hinge and introduce a unique XbaI site 3′ to the termination codon. The Fc fragment can be modified so that it is non-lytic (e.g., not able to activate the complement system). To make the non-lytic mutant IL-15 construct (we may refer to the non-lytic mutant as “mIL-15/Fc−−”), oligonucleotide site directed mutagenesis is used to replace the C1q binding motif Glu318, Lys320, Lys322 with Ala residues. Similarly, Leu235 is replaced with Glu to inactivate the FcγRI binding site. Ligation of cytokine and Fc components in the correct translational reading frame at the unique BamHI site yields a 1236 bp open reading frame encoding a single 411 amino acid polypeptide with a total of 13 cysteine residues. The mature secreted homodimeric IL-15/Fc−− is predicted to have a total of up to eight intramolecular and three inter-heavy chain disulfide linkages and a molecular weight of approximately 85 kDa, exclusive of glycosylation. Expression and Purification of mIL-15 Fc Fusion Proteins: Proper genetic construction of both mIL-15/Fc++, which carries the wild type Fcγ2a sequence, and mIL-15/Fc−− can be confirmed by DNA sequence analysis following cloning of the fusion genes as NotI-XbaI cassettes into the eukaryotic expression plasmid pRc/CMV (Invitrogen, San Diego, Calif.). This plasmid carries a CMV promoter/enhancer, a bovine growth hormone polyadenylation signal and a neomycin resistance gene for selection with G418 (of course, as noted above, many other plasmids are suitable as expression vectors; sequences encoding amino acid-based IL-15 and IL-15R antagonists can be placed under the control of other regulatory sequences; and one can select cells that carry the expression vectors, if desired, using any antibiotic resistance gene). Plasmids carrying the mIL-15/Fc++ or mIL-15/Fc−− fusion genes can be transfected into Chinese hamster ovary cells (CHO-K1 cells are available from the American Type Culture Collection) by electroporation (1.5 kV/3 μF/0.4 cm/PBS) and selected in serum-free Ultra-CHO™ media (Bio Whittaker Inc., Walkerville, Md.) containing 1.5 mg/ml of G418 (Geneticin, Gibco BRL). After subdloning, clones that produce high levels of the fusion protein can be selected by screening supernatants for IL-15 by ELISA (PharMingen, San Diego, Calif.). mIL-15/Fc fusion proteins are purified from culture supernatants by protein A Sepharose™ affinity chromatography followed by dialysis against PBS and 0.22 μm filter sterilization. Purified proteins can be stored at −20° C. before use.

Western blot analysis following SDS-PAGE under reducing (with DTT) and non-reducing (without DTT) conditions can be performed using monoclonal or polyclonal anti-mIL-15 or anti-Fcγ primary antibodies to evaluate the size and isotype specificity of the fusion proteins.

In studies of another cytokine, IL-2, we found that molecular weight (MW) measured by proteamic analysis could vary, depending upon the host cell type. The MW of IL-2/Fc produced by CHO cells was 94,838.7, while the same molecule produced in NS.1 cells was only 91,647.5. Differences in glycosylation may account for the difference in MW. Further, the difference in glycosylation appears to influence function, as IL-2/Fc molecules produced in CHO cells suppressed the development of diabetes in non-obese diabetic mice more effectively than the same molecule produced in NS.1 cells.

Standardization of the Biological Activity of Recombinant Mutant IL-15 and mIL-15/Fc−− proteins: Using the RT-PCR strategy and 5′ NotI sense oligonucleotide primer described above, mutant IL-15 cDNA with an XbaI restriction site added 3′ to its native termination codon, can be cloned into pRc/CMV. This construct can then be transiently expressed in COS cells (available from the American Type Culture Collection). The cells can be transfected by the DEAE dextran method and grown in serum-free UltraCulture™ medium (Bio Whittaker Inc.). Day 5 culture supernatant is sterile filtered and stored at −20° C. for use as a source of recombinant mutant IL-15 protein (rmIL-15). Mutant IL-15/Fc−− and mIL-15 mutant protein concentrations can be determined by ELISA as well as by bioassay, as described, for example, by Thompson-Snipes et al. (J. Exp. Med. 173:507, 1991). Dual probe ELISA assays are quantitative “sandwich” enzyme immunoassays. In one study, we coated microtiter plates with rat IgG antibodies specific for mouse/human IL-15. Test samples of IL-15/Fc were added to the wells, and unbound components in the sample were washed away. Enzyme-linked rabbit antibodies specific for mouse IgG2a Fc/human IgG1 Fc were then added to the wells, creating a sandwich, with IL-15/Fc bound by the coated anti-IL-15 antibody and the anti-mouse IgG2a Fc/human IgG1 Fc antibody. Such dual probe ELISAs ensure the assay is specific for mouse IL-15/Fc fusion protein (rather than IL-15 or mIgG2a/hIgG1). Excess enzyme-conjugated IgG can be removed by washing before the enzyme substrate is added to the wells. A colored reaction product develops in proportion to the amount of IL-15/Fc present in the sandwich.

The functional activity of mutant IL-15/Fc−− can be assessed by standard T cell proliferation assays, such as those described in U.S. Pat. No. 6,451,308. While a positive performance in a suitable assay (e.g., reduced lysis and therefore greater cellular proliferation, relative to a wild type IL-15 polypeptide, in a T cell proliferation assay) indicates that the Fc region within the fusion protein has been suitably modified, as noted, IL-15 and IL-15R mutants of the invention specifically include those that confer a clinical benefit on patients to whom they are administered (e.g., a patient who has received a heart, lung, or heart-lung transplant).

Determination of mIL-15/Fc−− or mIL-15/Fc++ Circulating Half-life: Serum concentrations of an IL-15 or IL-15R antagonist (e.g., fusion proteins containing a mutant IL-15 such as the mIL-5/Fc−− or mIL-5/Fc++ fusion proteins described above) can be determined over time following a single intravenous injection (or “bolus” injection) of the fusion protein (non-fusion proteins can be similarly assessed). Serial blood samples (as little as 100 μl may be required) can be obtained by standard methods at intervals of, for example, about 0.1, 6.0, 24.0, 48.0, 72.0, and 96.0 hours after administration of mutant IL-15/Fc−− protein. Measurements can employ an ELISA with a monoclonal antibody (e.g., a mIL-15 mAb) as the capture antibody. Where an Fc fusion protein is used, horseradish peroxidase conjugated to an anti-Fc antibody (e.g., an Fcγ2a mAb) can be used as the detection antibody. In such a configuration, the assay would be specific for the mutant IL-15/Fc−−.

Procedures for Screening IL-15 or IL-15R antagonists: One or more of the following transplantation paradigms and models of autoimmune disease can be employed to determine whether any given agent (e.g., any given mutant IL-15 polypeptide) is capable of functioning as an antagonist of IL-15 or of an IL-15R.

Antagonists, including those that contain a mutant IL-15 polypeptide can be administered in the context of well-established transplantation paradigms. Alternatively, where the antagonist is a polypeptide, one can administer a nucleic acid molecule encoding it. For example, a putative immunosuppressing polypeptide, or a nucleic acid molecule encoding it, can be systemically or locally administered by standard means to any conventional laboratory animal, such as a rat, mouse, rabbit, guinea pig, or dog, before an allogeneic or xenogeneic skin graft, organ transplant, or cell implantation is performed on the animal. Strains of mice such as C57B1-10, B10.BR, and B10.AKM (Jackson Laboratory, Bar Harbor, Me.), which have the same genetic background but are mismatched for the H-2 locus, are well suited for assessing various organ grafts.

A method for performing cardiac grafts by anastomosis of the donor heart to the great vessels in the abdomen of the host was first published by Ono et al. (J. Thorac. Cardiovasc. Surg. 57:225, 1969; see also Corry et al., Transplantation 16:343, 1973). According to this surgical procedure, the aorta of a donor heart is anastomosed to the abdominal aorta of the host, and the pulmonary artery of the donor heart is anastomosed to the adjacent vena cava using standard microvascular techniques (this procedure was used in the studies described below). Once the heart is grafted in place and warmed to 37° C. with Ringer's lactate solution, normal sinus rhythm will resume. Function of the transplanted heart can be assessed frequently by palpation of ventricular contractions through the abdominal wall. Rejection is defined as the cessation of myocardial contractions, which can be confirmed by examining the graft under anesthesia. IL-15 or IL-15R antagonists (e.g., mutant IL-15 polypeptides or fusion proteins containing them) would be considered effective in reducing organ rejection (or prolonging graft survival) if hosts that received the IL-15 or IL-15R antagonist tolerated the grafted heart longer than did untreated hosts. This model is typically carried out using a rodent, such as a mouse, but other animals can serve as models as well.

The effectiveness of IL-15 and IL-15R antagonists (e.g., mutant IL-15 polypeptides or fusion proteins containing them) can also be assessed following a skin graft. To perform a skin graft on a rodent, a donor animal is anesthetized and the full thickness skin is removed from a part of the tail. The recipient animal is also anesthetized, and a graft bed is prepared by removing a patch of skin from the shaved flank. Generally, the patch is approximately 0.5×0.5 cm. The skin from the donor is shaped to fit the graft bed, positioned, covered with gauze, and bandaged. The grafts can be inspected daily beginning on the sixth post-operative day, and are considered rejected when more than half of the transplanted epithelium appears to be non-viable. Skin grafts can be performed in animals other than rodents, including humans and non-human primates.

Models of autoimmune disease provide another means to assess IL-15 and IL-15R antagonists in vivo. These models are well known to skilled artisans and can be used to determine whether an agent, including any given mutant IL-15 polypeptide, would be therapeutically useful in treating a specific autoimmune disease when delivered to a patient (e.g., directly or via genetic therapy) or in prolonging graft survival or function.

The following materials and methods were used in the studies described below and can be used in connection with the compositions and methods described herein (for example, the animals and transplantation paradigms can be used in pre-clinical analysis of IL-15 or IL-15R antagonists, including mIL-15-containing fusion proteins).

Animals: BALB/c (H-2d) and C57BL/6 (H-2b) mice, 8-10 weeks old, were purchased from Charles River Laboratories (Wilmington, Mass.). B10.A (H-2d), CBA/Ca (H-2k), B10.BR (H-2k) and AKR/J (H-2k) mice were obtained from The Jackson Laboratory (Bar Harbor, Me.).

Reagents and Treatment Protocols: A construct (i.e., a vector) for expressing an IL-15 mutant/Fcγ2a fusion protein was designed as described by Kim et al. (J. Immunol. 160:5742, 1998). Glutamine residues 101 and 108 within the fourth alpha helix of IL-15 were mutated to aspartic acid via site-directed and PCR-assisted mutagenesis (see FIG. 3). This mutant IL-15 was then genetically linked to the hinge and constant regions of murine IgG2a and further cloned into an expression vector. NS.1 cells (obtained from ATCC), Manassas, Va.) or CHO-K1 cells (DMSZ, Braunschweig, Germany), were stably transfected with a plasmid carrying the construct encoding the fusion protein (Kim et al., J. Immunol. 160:5742, 1998). The transfected cells were cloned and cultured in serum-free Ultraculture™ media (Bio Whittaker Inc, Walkersville, Md.) containing 100 μg/ml Zeocin (Invitrogen, San Diego, Calif.). Fusion protein in the culture supernatant was purified by Protein A affinity chromatography and, in some instances, ion-exchange chromatography. A non-lytic IL-15 mutant/Fcγ2a fusion construct was generated essentially as described by Zheng et al. (see Zheng et al., J. Immunol. 163:4041, 1999, and Zheng et al., J. Immunol. 158:4507, 1997). Briefly, oligonucleotide site-directed mutagenesis was used to replace the IgG2a C1q binding motif Glu318, Lys320, Lys322 with Ala residues. Similarly, the IgG2a residue Leu235 was replaced with Glu to inactivate the FcγRI binding site (see Zheng et al., J. Immunol. 163:4041, 1999, and Zheng et al., J. Immunol. 158:4507, 1997).

A monoclonal antibody against CD154 (MR-1, IgG2a) was obtained from Chimerigen Laboratories (Allston, Mass.). Heart and islet allograft recipients were treated daily or every second day with 1.5 μg, 5 μg or 15 μg of the mutant IL-15-containing fusion protein by intraperitoneal injection or with 15 μg of control (IgG2a, also administered intraperitoneally) for a total of 14 days. The first treatment was given on the day of transplantation, after the surgical procedure. Treatment with anti-CD 154 (anti-CD40L) was with a single dose of 200 μg administered intraperitoneally on the day of transplantation, also after surgery had been completed.

Heart Transplantation: Abdominal heterotopic heart transplants were performed essentially as described by Corry et al. (Transplantation 16:343, 1973). The isolated donor heart was grafted by joining the donor aorta to the recipient aorta and the donor pulmonary artery to the recipient vena cava. After an initial recovery period, animals bearing such transplants were housed under standard conditions, and we recorded the palpable heartbeat of the graft every 1 to 2 days. Animals were scored as having rejected the graft upon complete loss of palpable heartbeat. In some instances, animals with long term surviving grafts received a secondary cervical heart transplant. The basic procedures were identical to the ones used for abdominal aortic grafts, except that the second heart was grafted onto the carotid artery by side to end anastomosis with the aorta and side to end anastomosis of the pulmonary artery to the jugular vein. In all instances, 11-0 suture material was used for these procedures.

Islet transplantation: Islet transplantation was performed according to procedures described by Ferrari-Lacraz et al. (J. Immunol. 167:3478, 2001). Donor pancreata from 8-10 wk male Balb/c (H-2d) mice were perfused in situ with 4 ml Type IV collagenase (Worthington Biochemical Corp. Freehold, N.J.) through the common bile duct. The pancreata were harvested after perfusion and incubated at 37° C. for 35 minutes. Islets were released from the pancreata by gentle vortexing and further purified on discontinuous percoll gradients, washed twice and 300 to 400 islets were transplanted under the left renal capsule of 8-10 wk old, completely MHC mismatched, C57BL/6 recipients rendered diabetic by a single intraperitoneal injection of streptozotocin (260 mg/kg in 0.9% NaCl; Sigma Chemical Co., St. Louis, Mo.). Allograft function was monitored by serial blood glucose measurements (Accu-Chek™ III blood glucose monitor; Boehringer Mannheim, Indianapolis, In.). Primary graft function was defined as a blood glucose level below 200 mg/dl on day 3 post-transplantation, and graft rejection was defined as a rise in blood glucose exceeding 300 mg/dl following a period of satisfactory primary graft function. To determine whether tolerance was evident in the treated population, a nephrectomy was performed on islet allograft recipient mice with euglycemia for 120 days after primary transplantation. Removal of the left kidney bearing the islet allograft 120 days post-transplantation resulted in prompt hyperglycemia exceeding 300 mg/dl within 2-3 days. The second islet allografts from Balb/c or B10.A donors were transplanted under the right kidney capsule of hyperglycemic mice 4-6 days post nephrectomy. We monitored secondary graft function by measuring the blood glucose levels of the recipient mice as described above.

Histopathology and Immunochistochemistry: Transplanted hearts were harvested at Day 5 after transplantation and divided into three parts by cutting through the heart twice, perpendicular to the intraventricular septum. The first 1/3 of the tissue was fixed in zinc formalin for hematoxylin/eosin and immunohistochemistry (CD3 and F4/80 detection), and paraffin sections were prepared from these samples; the second ⅓ of the tissue was imbedded in OCT and snap-frozen in liquid nitrogen to −80° C. for immunohistochemistry (CD4 and CD8 detection); and the last ⅓ was analyzed by RT-PCR (see below). After dehydration and paraffin embedding, 5- to 6-μm-thick sections of the heart were stained with H&E. Multiple sections of each heart were prepared and examined for the extent of rejection, myocardial damage, mononuclear cell infiltration, vasculitis and intimal proliferation. The avidin-biotin immunoperoxidase method was used for immunohistochemistry. Images were obtained using an Axioscope™ 2 microscope (Zeiss) equipped with a digital camera (SV Micro 80155) and interfaced with image analysis software (KS 300). Quantitative image analysis was performed on ten random sections from each section of the heart stained for different cell markers (CD4 and CD8). Quantitative image analysis was performed on three hearts from the control group and three hearts from the treatment group. The number of positively stained cells and total area occupied by these cells were compared for CD4 and CD8 cell markers in hearts of treated and control animals.

For islet transplants, the left kidneys bearing islet allografts were removed from long-term graft accepting mice and processed further. In addition, transplant-bearing kidneys from C57Bl/6 mice that had received Balb/c islet allografts were removed on Day 7 post-transplantation. The kidneys were fixed in zinc formalin for hematoxylin/eosin and aldehyde-fuchsin staining and immunohistochemistry (insulin detection); paraffin sections were prepared from the samples processed in this way, and 5- to 6-μm-thick sections of areas of islet implantation were stained. Multiple sections of each kidney were prepared and examined for islet content and insulin production. The avidin-biotin immunoperoxidase method was used for immunohistochemistry, and images obtained as described for heart transplants.

RNA isolation and reverse transcriptase assisted polymerase chain reaction (RT-PCR): Total cellular RNA was extracted using RNASTAT™ 60 (Tel Test, Friendswood, Tex.) according to the manufacturer's instructions. We checked the quality of the RNA by performing a PCR analysis to detect traces of chromosomal DNA, and we determined the concentration of the RNA using a Beckman Coulter Spectrophotometer DU 640. Two micrograms of RNA were reverse-transcribed and quality controlled for the expression of the housekeeping gene cyclophilin (Smith et al., J. Immunol. 165:3444, 2000). Subsequently, the relative abundance of the inflammatory cytokines (IL-1β, IL-6 and TNFα), IFNγ, and the CTL markers FasL, granzyme B and perforin were determined by TaqMan™ real-time PCR analysis with the ABI 7000 Sequence detection instrument and normalized against the housekeeping gene cyclophilin. Primers and probes for IL-1β, IL-6 and TNFα were purchased from Applied Biosystems, primers for cyclophilin (CYC), IFNγ (IFN), FasL (FSL), granzyme B (GRB) and perforin (PRF) were:

| | | |
|---|---|---|
| CYCF: | GCCTGGATGCTAACAGAAGGA; | (SEQ ID NO:11) |
| CYCR: | GTTCATCCCGTCGCTATGGT; | (SEQ ID NO:12) |
| CYCprobe: | ATGACAAGGATGCCGGGCAAGTGT; | (SEQ ID NO:13) |
| FSLF: | AATCTGTGGCTACCGGTGGTA; | (SEQ ID NO:14) |
| FSLR: | GGTGGAAGAGCTGATACATTCCTA; | (SEQ ID NO:15) |
| FSLprobe: | ATGGTTCTGGTGGCTCTGGTTGGAA; | (SEQ ID NO:16) |
| GRBF: | GCAAAGACTGGCTTCATATCCAT | (SEQ ID NO:17) |
| GRBR: | GCAGAAGAGGTGTTCCATTGG; | (SEQ ID NO:18) |
| GRBprobe: | ACAAGGACCAGCTCTGTCCTTGGCAG; | (SEQ ID NO:19) |
| PRFF: | TGCTCTTCGGGAACCAAGCT; | (SEQ ID NO:20) |
| PRFR: | CAGGGTTGCTGGGCAGTGA; | (SEQ ID NO:21) |
| PRFprobe: | CACCAGAGCAGTTCTCAACCTGGAC | (SEQ ID NO:22) |
| | AGC; | |
| IFNF: | ACAATGAACGCTACACACTGCAT; | (SEQ ID NO:23) |
| IFNR: | TGGCAGTAACAGCCAGAAACA; | (SEQ ID NO:24) |
| IFNprobe: | TTGGCTTTGCAGCTCTTCCTCATGG. | (SEQ ID NO:25) |

Statistical analysis: Animal survival data were analyzed using a survival curve Logrank test as provided by Prism™ software (version 3.0). Histological data generated by Image Analysis were evaluated for statistical significance using Student's two-tailed t test at the 0.05 significance level. The Microsoft Excel data analysis tool was used to obtain mean and standard deviation as well as Student's t test results. We generated real-time PCR data by analyzing each cDNA sample in triplicate by TaqMan™ realtime PCR. Automatic baseline determination using the ABI 7000 Sequence detection instrument was followed by manual quality control. Primary data were processed in an Excel spreadsheet format and exported into the Prism software (version 3.0) for the graphical display. Data generated were evaluated for statistical significance using a Student's two tailed t test.

As noted above, we have found that administration of a lytic and antagonistic IL-15 mutant/Fcγ2a fusion protein can prevent rejection and induce antigen-specific tolerance of minor histocompatibility complex-mismatched grafts in a B10.Br to CBA/Ca strain combination. This fusion protein can also prolong the survival of transplanted hearts in fully MHC-mismatched recipients, as we demonstrated with a Balb/c to C57BL/6 mouse strain combination. Prolonged graft survival was accompanied by reduced mononuclear cell infiltration and inflammatory cytokine expression in the treated graft recipients. In addition, we found that administering the fusion protein in combination with a sub-optimal dose of anti-CD 154 (CD40L) antibody confers permanent heart allograft engraftment in a fully MHC-mismatched mouse strain combination. Moreover, we demonstrated that an IL-15 mutant/Fcγ2a fusion protein is capable of inducing antigen-specific tolerance in a fully MHC-mismatched islet transplant model.

To further characterize the antagonist's mode of action, we performed parallel experiments employing a variant with a point-mutated non-lytic IgG2a Fc. These experiments demonstrated that the Fc portion contributes to the overall efficacy of the molecule in vivo.

Treatment with IL-15 mutant/Fcγ2a fusion protein prolongs the survival of fully MHC-mismatched heart allografts.

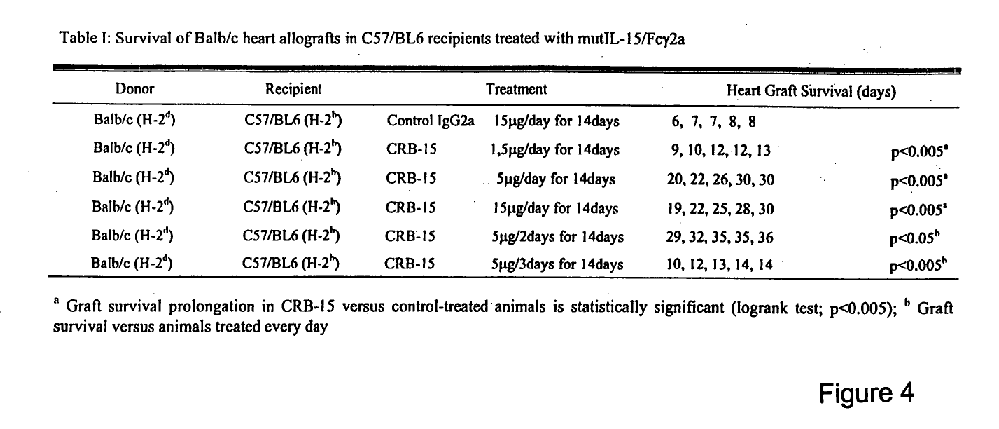

We tested the efficacy of the fusion protein in preventing the rejection of fully MHC-mismatched heterotopic heart transplants in the Balb/c (H-2d) to C57BL/6 (H-2b) mouse strain combination. Control animals rejected the transplants with an MST=7d (Table I). While recipient C57BL/6 mice treated with 1.5 μg of IL-15 mutant/Fcγ2a fusion protein daily (for 14 days) experienced a marginal prolongation of engraftment, treatment with 5 μg of the fusion protein daily (again, for 14 days) resulted in a pronounced prolongation of graft survival (MST=26d). In contrast, treatment with 15 μg did not lead to a further prolongation of graft survival, and animals in this treatment group rejected their transplants with kinetics similar to the animals in the 5 μg dose group (Table I). Interestingly, treatment of transplant recipients with 5 μg every second day for 14 days (8 administrations total) led to a further prolongation of graft survival with an MST=35d (Table I). Treatment with 5 μg every three days (5 administrations total) showed an accelerated rejection of the transplanted hearts, as compared to a daily or bi-daily treatment regimen (Table I, shown as FIG. 4).

To assess the effect of IL-15 mutant/Fcγ2a fusion protein on graft rejection, we studied the graft cellularity in heart allografts harvested 5 days post-transplantation. The overall graft cellularity in treated mice was reduced compared to the control group. The inflammatory infiltrates in these hearts were focal, less numerous, and smaller than in the control-treated animals and ischemic myocardial cell damage with interstitial edema and hemorrhages was also strongly reduced in the treated animals. Vascular changes consisting of vasculitis and vascular endothelial cell proliferation and occlusion were also more evident in the control group than in the allografts of treated animals. A quantitative image analysis performed on these samples revealed a particularly striking reduction of leukocyte infiltration for CD8+ T cells, which was at 93.5% (n=3, p=0.008). Immunohistological detection of leukocyte subsets on day 5 showed a strongly reduced number of CD3+, CD4+, CD8+, and F4/80+comparison, CD4+ T cells in the treated grafts were reduced by 58% (n=3, p<0.05).

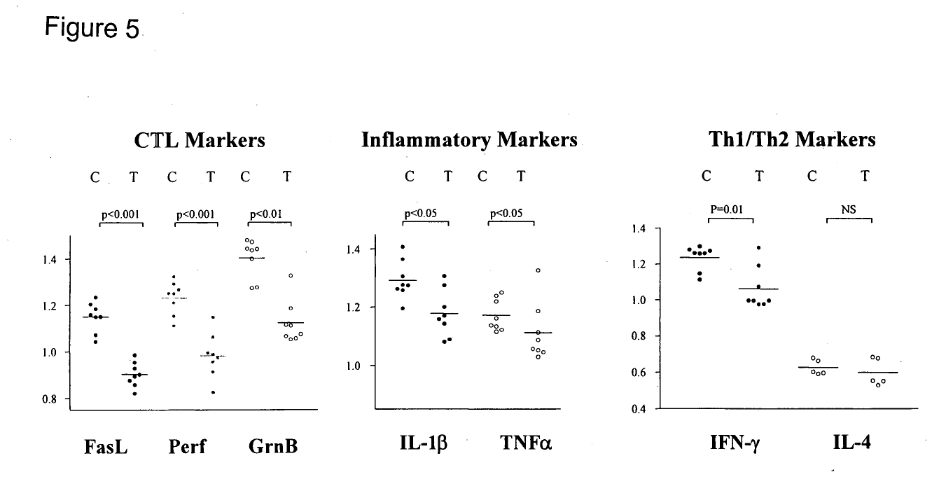

To further study the effects of treatment on allogeneic transplant rejection, a real time PCR analysis on various inflammatory cytokines (IL-1β and TNFα), CTL effector molecules (FasL, Granzyme B and Perforin) and Th1/Th2 cytokines (IL-4 and IFNγ) was performed 5 days post transplantation. Whereas the expression of all of these markers was elevated in rejecting heart allografts of control-treated animals (C), treatment with IL-15 mutant/Fcγ2a fusion protein (T) led to a statistically significant reduction of expression of most of these genes in the transplanted hearts, with the notable exception of the Th2 cytokine IL-4 (FIG. 5). Similar results such as for IL-4 were also obtained for IL-5. Interestingly, a reduction in IL-10 expression was also observed in the treated grafts (p<0.001), likely reflecting the strong reduction in macrophages seen in the treated grafts. The Fc portion contributes to the efficacy of IL-15 mutant/Fcγ2a fusion protein in vivo.

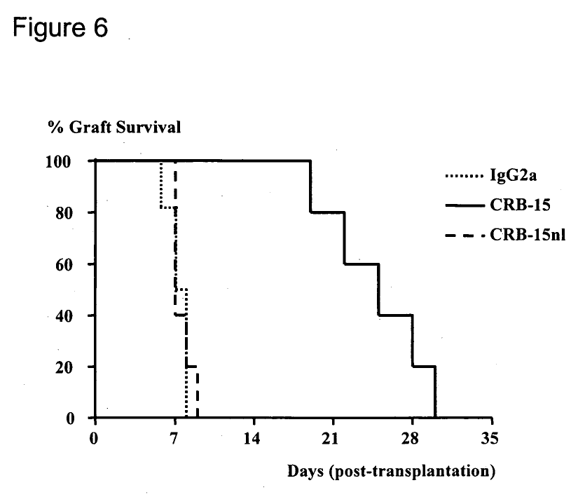

Earlier studies have demonstrated that the deletion of antigen-specific T cells contributes to long-term engraftment and tolerance induction in various allograft settings (Li et al., Nat. Med. 5:1298, 1999; Wells et al., Nat. Med. 5:1303, 1999). To further characterize the antagonists's mode of action and to directly investigate the potential contribution of the IgG2a Fc terminus to the overall efficacy of the fusion protein, we generated a non-lytic point-mutated variant (IL-15nl) that does not interact with complement or Fc receptors. Whereas a short course treatment with the lytic fusion protein leads to prolonged heart allograft survival (MST=25 days) in the Balb/c to C57/BL6 mouse strain combination, transplants in animals treated with the non-lytic variant IL-15nl are rejected with kinetics comparable to control-treated animals (MST=7 days)(FIG. 6).

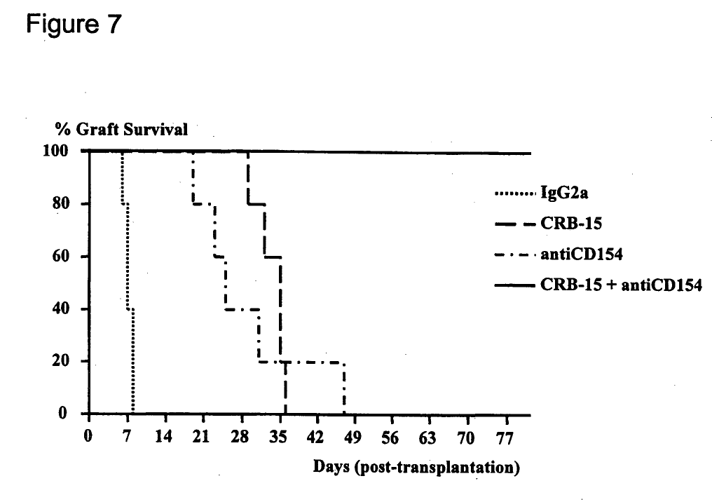

Permanent engraftment of MHC-mismatched allografts after treatment with IL-15 mutant/Fcγ2a fusion protein and a single dose of anti-CD154 antibody. While the treatment we administered prolongs heart allograft survival in MHC-mismatched recipients, the transplants are eventually rejected (Table I). As we have shown that treatment can prevent costimulation blockade resistant rejection in islet transplant models (Ferrari-Lacraz et al., J. Immunol. 167:3478, 2001), we were interested in determining whether blockade of the CD40/CD 154 costimulation pathway would synergize with our lytic IL-15 antagonist in preventing heart allograft rejection. Whereas treatment with a single dose of the anti-CD 154 monoclonal antibody MR-1 prolonged heart transplant survival in the Balb/c to C57/BL6 mouse strain combination, this treatment was insufficient to prevent rejection (FIG. 7). In contrast, treatment with a lytic IL-15 fusion protein (5 μg/mouse every 2nd day) for 14 days, in combination with a single dose administration of the anti-CD154 antibody, was sufficient to prevent graft rejection in all animals tested (n=5) and led to permanent engraftment of the transplanted hearts (FIG. 7). IL-15 mutant/Fcγ2a fusion protein as a monotherapy can induce antigen-specific tolerance.

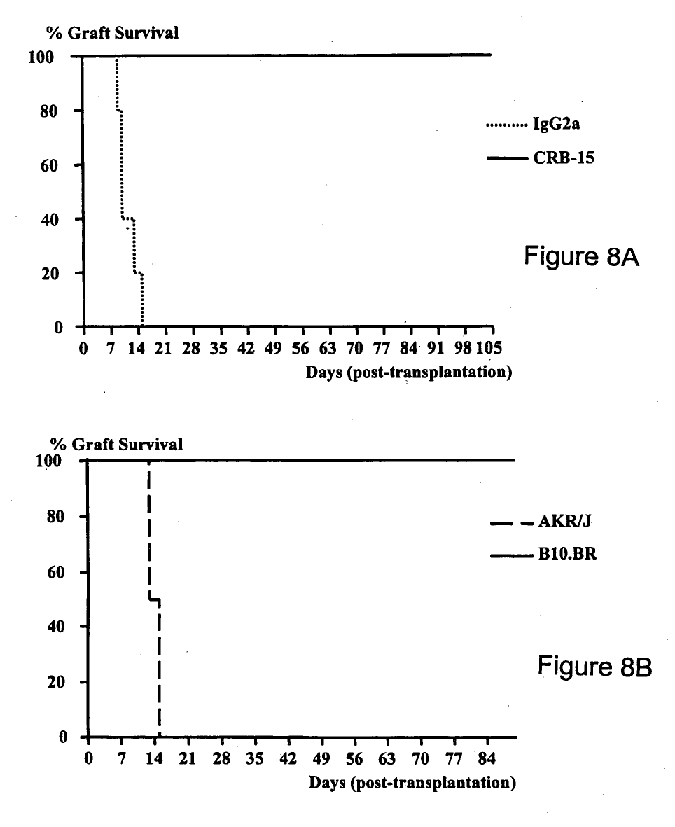

To further explore the therapeutic potential of such antagonists, the efficacy of the fusion protein in preventing the rejection of heterotopic heart transplants in a minor histocompatibility mismatch strain combination was tested by transplanting hearts from B10.BR to CBA/Ca mice. Treatment with 5 μg administered every second day for 14 days led to permanent engraftment of the transplanted hearts in this mouse strain combination. Control hearts were all rejected within 13 days after transplantation (MST=10 days)(FIG. 8A). To test for antigen-specific tolerances, the CBA.Ca mice having received B10.BR allografts, and having been treated received secondary heart allografts after prolonged survival of the primary grafts (>100 d). These secondary heart transplants were from either B10.BR mice or from the third party strain AKR.J. While the secondary grafts from the B10.BR donors were accepted without any further immunosuppression, the grafts from the AKR.J mice were efficiently rejected (FIG. 8B).

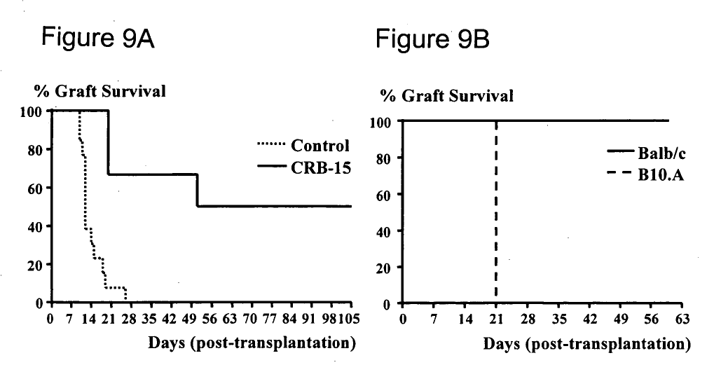

Similarly, administration of IL-15 mutant/Fcγ2a fusion protein proved efficacious in preventing the rejection of islet allografts transplanted under the kidney capsule of streptozotocin-induced diabetic mice in the fully MHC-mismatched Balb/c to C57/BL6 strain combination. Treatment with 5 μg of the fusion protein administered every second day for 14 days prolonged islet allograft survival and permanent engraftment in 50% of the treated animals (FIG. 9A). Seven days after transplantation, a strong reduction in islet cell mass and insulin-producing cells was apparent in untreated animals, as compared to the treated mice. 120 days after transplantation, the graft containing kidneys were removed from the treated animals with permanent engraftment and grafted islets in these animals were found to be preserved and functional, as determined by aldehyde-fuchsin and insulin staining. All animals examined became diabetic after removal of the grafts. Subsequently, these animals received a second islet graft under the capsule of the second kidney. Whereas mice receiving islets from Balb/c donors became normoglycemic, and remained so without any further treatment, a B10.A derived graft was rejected (FIG. 9B). We conclude that this treatment protocol can lead to antigen-specific tolerance and that IL-15 mutant/Fcγ2a fusion protein monotherapy has the potential to induce tolerance also in a fully MHC-mismatched allograft setting.

The present studies extend prior observations by showing that treatment with an IL-15 antagonist also prolongs the graft survival of fully MHC-mismatched vascularized heart transplants. We find that treatment reduces the graft infiltration by CD4+ and CD8+ T cells as well as macrophages. The effect of treatment is particularly striking for CD8+ T cells, in that CD8+ T cells are almost completely absent from the grafts of treated animals. In comparison, the effect on CD4+ T cells appears to be more moderate, a finding that is not surprising in view of earlier reports that IL-15 acts preferentially on CD8+ T cells, at least in IL-15,and IL-15Rα knockout systems (Lodolce et al., Immunity 9:669, 1998; Kennedy et al., J. Exp. Med. 191:771, 2000).

Consistent with the immunohistology results, we find that treatment with an IL-15 antagonist reduces the expression of CTL markers in the grafts, as well as the expression of the inflammatory cytokines TNFα and IL-1β. Interestingly, treatment with the fusion protein leads to a reduction of Th1 cytokine expression (IFNγ and TNFα), but has no effect on the expression of the Th2 cytokines IL-4 and IL-5. These data indicate that IL-15 may preferentially stimulate Th1 responses, further underlining the utility of IL-15 antagonistic approaches in targeting Th1-mediated diseases, such as many autoimmune disorders and graft rejection. The dose titration experiments performed in the Balb/c to C57/BL6 mouse strain combination revealed a dose response relationship and a direct correlation between the dose administered and the efficacy of the treatment. Interestingly, treatment every second day showed an increased efficacy as compared to a daily treatment and further delayed graft rejection. Although not further examined, one possible explanation for this observation would be that IL-15/IL-15R signaling within the tissue might be protective under conditions of ischemia and/or reperfusion, such as in the initial periods post surgery.

The reduced efficacy we observed when the fusion protein is administered only once every three days, on the other hand, is consistent with the observed half-life of the molecule in mice, which is about 30 hours.

We have previously demonstrated that the deletion of activated T cells can contribute to peripheral tolerance induction, suggestive of the notion that depletion of the pool of antigen-responsive T cells may shift the balance of an immune reaction from an immunogenic to a tolerogenic response (Li et al., Immunity 14:407, 2001). In view of these earlier findings we were interested in determining whether the IgG2a Fc portion of the fusion protein tested would contribute to the overall efficacy of the molecule. Intriguingly, we find that the treatment with a non-lytic variant did not prolong graft survival in the MHC mismatch transplant model. These results suggest that complement and/or FcR mediated deletion of IL-15R expressing activated T cells and macrophages contributes to the overall immunoprotective effect of the lytic, antagonistic fusion protein. Interestingly, Smith et al. reported earlier that the use of a recombinant soluble IL-15R alpha subunit (sIL-15Rα) was ineffective in preventing graft rejection in the MHC mismatch heart transplant model, but did prolong graft survival in a minor histocompatibility mismatch mouse strain combination. An IL-15 neutralizing agent, such as sIL-15Rα, would not target IL-15R bearing cells for deletion by the innate immune system. We would therefore propose that while inhibition of the IL-15/IL-15R pathway is sufficient to prevent graft rejection and induce antigen-specific tolerance in a minor histocompatibility mismatch mouse heart transplant setting, Fc-mediated activation of the innate immune system and depletion of IL-15R bearing cells contributes to the prolonged graft survival of fully MHC mismatched heart transplants observed in this study.

In addition to prolonging graft survival, we find that a short course of treatment can induce antigen-specific tolerance in both, minor histocompatibility mismatched heart transplants, as well as in fully MHC-mismatched islet allografts. Furthermore, the fusion protein synergizes with the costimulation blocker anti-CD 154 in preventing heart transplant rejection.

A number of embodiments of the invention have been described. Nevertheless, it will be understood that various modifications may be made without departing from the spirit and scope of the invention. Accordingly, other embodiments are within the scope of the following claims.

What is claimed is:

1. A method of suppressing an IL-15-dependent immune response, the method comprising:
(a) providing a patient who has experienced, or who is at risk for experiencing, an IL-15-dependent immune response; and
(b) administering to the patient a physiologically acceptable composition comprising a polypeptide comprising SEQ ID NO:6 or a nucleic acid sequence encoding a polypeptide comprising SEQ ID NO:6, wherein the amount of the composition is sufficient to suppress the IL-15-dependent immune response.

2. The method of claim 1, wherein the patient has received, or is scheduled to receive, a transplant comprising an organ or biological tissue.

3. The method of claim 2, wherein the transplant originated in a donor having a complete or partial immunological incompatibility with the patient.

4. The method of claim 2, wherein the transplant comprises a heart.

5. The method of claim 2, wherein the transplant comprises a kidney.

6. The method of claim 2, wherein the transplant comprises tissue of the skin, liver, or lung.

7. The method of claim 2, wherein the patient is a human patient.

8. The method of claim 1, further comprising administering to the patient an agent that inhibits CD40L.

9. The method of claim 8, wherein the agent that inhibits CD40L is an anti-CD154 antibody.

10. The method of claim 1, wherein the patient has, or is at risk of developing, an autoimmune disease.

11. The method of claim 10, wherein the autoimmune disease is rheumatoid arthritis.

12. The method of claim 1, wherein the patient has, or is at risk of developing, vasculitis.
